(19) United States
(12) Patent Application Publication
(10) Pub. No.: US 2012/0302492 A1
(43) Pub. Date: Nov. 29, 2012

(54) Antibody Mimetic Scaffolds

(75) Inventors: Richard Harkins, Alameda, CA (US); Fang Jin, Waban, MA (US); Ye Jin, Danville, CA (US); Marian Seto, Orinda, CA (US)

(73) Assignee: BAYER HEALTHCARE LLC, Tarrytown, NY (US)

(21) Appl. No.: 13/504,775

(22) PCT Filed: Nov. 1, 2010
(86) PCT No.: PCT/US10/55014

§ 371 Date: Apr. 27, 2012

Related U.S. Application Data
(60) Provisional application No. 61/256,901, filed on Oct. 30, 2009.

(51) Int. Cl. C07K 14/435; C12N 15/85; A61K 38/17; C40B 40/10; C40B 40/06; C12N 15/12; C12N 5/10

(52) U.S. Cl. 514/1.1; 536/23.5; 435/320.1; 435/358; 506/18; 506/16; 530/350

(57) ABSTRACT

Provided herein are protein scaffolds, e.g. antibody mimetic scaffolds, comprising a three finger protein domain that specifically bind to target molecules, polynucleotides encoding such proteins, methods of using such proteins, and libraries of such scaffolds.

### REFERENCE TO RELATED APPLICATIONS

This application claims priority to U.S. Provisional Application Ser. No. 61/256,901 filed on Oct. 30, 2009 and is hereby incorporated by reference for all purposes.

### SEQUENCE LISTING

This application is being filed with a sequence listing in both paper and electronic format. Applicants certify that the contents of the paper and electronic versions are identical.

### FIELD

Provided herein are protein scaffolds and libraries thereof useful for the generation and screening of products having novel binding characteristics.

### BACKGROUND

Hybridoma and recombinant DNA technologies have led to the development of several highly successful therapeutic monoclonal antibody drugs. Monoclonal antibodies (mABs) represent a major segment of the $100 billion Biologics market and are expected to be the key driver for the growth of Biologics over the next decade. However, despite the progress and success, mAbs as a Biologics class are expensive and difficult to manufacture due to their size (150 kDa) and complexity (multimeric, glycosylated heavy and light chains). Functionally, mABs display poor tissue penetration and the presence of the Fc region can result in undesired Fc receptor interactions and complement activation. To circumvent these issues smaller alternative antibody and non-antibody based formats, called antibody mimetics, have been developed that are much easier to engineer and produce. Antibody mimetics have been shown to display equal or superior binding affinity and specificity compared to traditional antibodies with additional effector functions that can be chemically or genetically included depending upon the target indication.

Several antibody mimetics scaffolds have been engineered to bind therapeutic targets with potencies and selectivities that match that of traditional antibodies. Antibody mimetics have been developed utilizing an immunoglobulin-like fold, for example, fibronectin type III, NCAM and CTLA-4. Other mimetics scaffolds bearing no similarity to immunoglobulin folds have been successfully validated and are in either preclinical or clinical development.

The “three finger” fold was first identified in snake venom neurotoxins from Elapids (sea snakes and cobras), and over 275 sequences have been identified in this large multigene superfamily, all of which share common structural features. Despite their similar structural properties, the three finger toxins bind diverse molecular targets with exquisite selectivity and high potency. For example, short and long chain neurotoxins bind the α1 nicotinic actylcholine receptor (AChR), an ion channel, the muscarinic toxins bind the muscarinic AChR, a G-coupled protein receptor (GPCR), dendroaspin and cardiotoxin A5 target the integrins, aIIbβ3 and αvβ3, respectively, and calciseptine and FS2 toxin bind the L-type calcium channel. In mammals, the TFPDs occur mainly as cell surface proteins that exhibit diverse binding properties that includes serving as receptors for ligands such as urokinase (urokinase receptor), TGF-β, activin, and bone morphogenic protein (TGF-β receptor family), and complement proteins C8 and C9 (CD59).

### SUMMARY

Provided herein are protein scaffolds, for example antibody mimetic scaffolds, comprising a three finger protein domain that specifically bind to target molecules, polynucleotides encoding such proteins, and methods of using such proteins for example for the treatment and/or diagnosis of human diseases. Also provided are libraries of such scaffolds.

Accordingly, provided is a polypeptide comprising a three finger protein domain (TFPD) wherein said TFPD has an amino acid sequence that has been modified relative to the amino acid sequence of a naturally occurring TFPD such that the modified TFPD binds to a specified target molecule. In some embodiments, the target molecule bound by the modified TFPD is not bound by the naturally occurring TFPD. In some embodiments, the modification is at a position in finger 1 (F1) of said TFPD, finger 2 (F2) of said TFPD, finger 3 (F3) of said TFPD, or a combination of two or more fingers selected from the group consisting of F1, F2, and F3. Such modifications may be substitutions or insertions, and further may be specifically engineered or randomly generated to cover a broad range of possible target molecules.

In some embodiments, the TFPD is a mammalian TFPD. In some embodiments, the TFPD is a human TFPD. In some embodiments, the human TFPD is selected from the group of genes within the Ly-6/uPAR (LU) protein domain family consisting of CD59, urokinase receptor (uPAR) domain 1, uPAR domain 2, uPAR domain 3, the TGF-β receptor family of TFPDs, including TGFR domain 1, TGFR domain 2, ACVR1, ACV1B, ACV1C, ACVL1, AMHR2, AVR2A, AVR2B, BMR1B, BMP1A, BMPR2, members of the human uPAR gene cluster including LYPD3-1, LYPD3-2, LYPD4-1, LYPD4-2, LYPD5-1, LYPD5-2, TX101-1, TX101-2, CD177-1, CD177-2, CD177-3, CD177-4, and additional human genes in the LU family containing TFPDs including LYPD1, LYPD2, LYPD6, LPD6B, LY6E, LY6D, LY6DL, LY66C, LY6K, LYG6E, LY65C, LY65B, LY66D, LY6H, LYNX1, PATE, PATEB, PATEDJ, PATEM, PSCA, SLUR1, SLUR2, ASPX, HDBP1, SACA4, C9orf57, and BAMBI. Additionally, in some embodiments, the TFPD is an inactive mutant.

Additionally, provided are libraries of polypeptides comprising a plurality of three finger protein domains (TFPD) wherein said TFPD have amino acid sequences that has been modified relative to the amino acid sequences of corresponding naturally occurring TFPD such that the modified TFPD binds to a specified target molecule.

Also provided is a method of using a three fingered protein domain (TFPD) as a scaffold for generating antibody mimetics comprising inserting an amino acid sequence into one or more fingers of the TFPD. In some embodiments, the TFPD is CD59 or uPAR domain 3. In some embodiments, the inserted amino acid sequence is a random sequence.

Also provided are polynucleotides encoding for the polypeptides disclosed herein, and vectors and host cells containing such polynucleotides.

### DESCRIPTION OF THE DRAWINGS

FIG. 1 shows an alignment of human TFPD sequences. Fifty three human TFPD containing sequences were obtained from the UniProt database and aligned. CD59 and uPAR domain 3 are indicated by the arrows.

FIG. 2 shows the conservation of TFPD topology across species. The structures for snake venom α, human CD59, and human uPAR domain 3 were obtained from the worldwide protein database (pdb accession numbers liq9, 2uwr, and 2fd6 respectively). The backbone polypeptide chains are shown and the three “fingers” or loops (F1, F2, and F3) are shown for each structure.

FIG. 3 shows a human TFPD sequence diversity plot. Seventeen human TFPD sequences that code for soluble proteins or extracellular domains of cell surface receptors were aligned, and a consensus sequence obtained. The location of the sequence relative to the TFPD three fingers is shown. The sequence diversity at each position is expressed as shown by a Wu-Kabat protein variability plot.

FIG. 4 shows sequence alignment across species for two TFPDs, CD59 and uPAR domain 3. FIG. 4(A) shows sequence alignments for CD59 and FIG. 4(B) shows sequence alignments for uPAR D3 across several species. The SwissProt/UniProt ID's denote the following organisms: HUMAN, human; AOTTR, owl monkey; CALSQ, marmoset; CERAE, African green monkey; MACFA, crab-eating macaque; PAPSP, baboon; PONAB, orangutan; SAISC, squirrel monkey; PANTR, chimpanzee; RABIT, rabbit; PIG, pig: RAT, rat; MOUSE, mouse.

FIG. 5 shows a design of hCD59 RGD insertion variants. The RGD containing sequences from eristostatin, RGD1, RGD2, and RGD3, consisting of 7, 9, or 11 residues respectively, were engineered into the tips of each finger of hCD59 as shown. The insertion points within each finger are indicated by circled residues whereby the inserted residues were substituted for the circled residues.

FIG. 6 shows expression and functional analysis of hCD59 and its RGD loop insertion variants. A 1 ml aliquot of HB2151 cells carrying flag tagged wtCD59 or its RGD loop insertion variants was harvested after 3-4 hours of IPTG induction with the OD₆₀₀ reaching around 2. From this, 100 μl of periplasmic fraction was extracted from every 1 ml of HB2151 cell. Shown in FIG. 6A are periplasmic fractions from E. coli expressed wtCD59 or CD59 F2 RGD1, RGD2, and RGD3 variants separated on 4-12% Bis-Tris gel wherein the protein expression was detected by HPR anti-flag monoclonal antibody. Each lane is loaded with 3.25 μl periplasmic fraction, with Trefoil Factor 1 (TFF1) as a control. Shown in FIG. 6B are graphs of functional analysis of the CD59 F2 loop insertion variants as measured by C9 or GPIIb/IIIa binding assay. FIGS. 6C and 6D show the expression (C) and functional analysis (D) of hCD59 RGD insertional variants in the F1 and F3 loops.

FIG. 7 shows the expression of uPAR domain 3 (uPARD3) and its F2 RGD loop insertion variants, and testing their binding capacity to GPIIb/IIIa. FIG. 7A shows expression of flag tagged wt uPARD3 and uPARD3 F2 variants in HB2151 periplasmic fractions, as detected by an anti-flag antibody. FIG. 7B shows the GPIIb/IIIa binding assay for the uPARD3 F2 RGD insertion variants. TFF1 is used as a negative control.

FIG. 8 shows competitive binding ability of hCD59 F2 RGD variant to GPIIb/IIIa. Serially diluted (1:5 dilution stepwise) competitors RGD peptide Integrilin and fibrinogen are pre-incubated with 2.5 μL1 of CD59/F2/RGD3 periplasmic fraction and then added to GPIIb/IIIa-coated 96-well plates, followed by the same procedure as the GPIIb/IIIa binding assay. As a negative control, 2.5 μl of wtCD59 is used.

FIG. 9 shows western blot analysis to demonstrate the display of the CD59-pIII fusion protein on the surface of the phage particles. 2×10⁹ phage particles bearing wtCD59 or its RGD variants or M13K07 control phage were separated on 4-12% Bis-Tris gel. The display of CD59 on the surface of phage was probed by anti-pIII monoclonal antibody and anti-flag antibodies.

FIG. 10 shows graphs displaying functional analysis of phage particles bearing wtCD59 or its RGD variants. The binding ability of phage wtCD59 or its RGD variants to C9 or IIb/IIIa were measured by C9 or GPIIb/IIIa binding assay.

FIG. 11 shows competitive binding ability of phage CD59 F2 RGD variant to GPIIb/IIIa. Serially diluted (1:10 dilution stepwise) competitors RGD peptide Integrilin, and fibrinogen and negative control KG were pre-mixed with phage CD59/F2/RGD1 and then added to GPIIb/IIIa-coated 96-well plate, followed by the same procedure as GPIIb/IIIa binding assay. Phage wtCD59 was used as negative control.

FIG. 12 depicts CD59/F2/RGD1 phagemid DNA analysis following A) C9 and B) GPIIb/IIIa binding assay. Equal amounts of phage wtCD59 and phage CD59/RGD1 were pre-mixed and incubated with C9-coated or IIb/IIIa-coated plates. After washing away unbounded phage, the bounded phage were eluted with 10 mM glycine pH2.8 and neutralized with 1M Tris buffer pH8.0. Then the exponentially growing TG1 cells were infected with the eluted phage, plated on 2xYT agar plate with ampicillin and incubated overnight at 30° C. Twelve clones were randomly picked from each plate originated from C9-coated or GPIIb/IIIa-coated phage elutes. Phagemid DNA was isolated and analyzed by PstI or DraI11 digestion. The wells with odd number are PstI digestions while the wells with even number are DraI11 digestion.

FIG. 13 depicts a table showing sequence analysis of CD59/F2/7-mer library. Over ninety clones were sequenced from the CD59/F2/7-mer library. This table illustrates the frequency of each amino acid (out of 20 amino acids) at each of the 7 positions for 69 in-frame sequences.

FIG. 14 shows western analysis of CD59 protein expressed from the F2 7-mer random library. Twenty-two (22) individual clones expressing CD59/F2/7-mer in HB2151 cells were picked and induced with IPTG. Periplasmic fractions were isolated and separated on 4-12% Bis-Tris gels. The soluble proteins were detected with HRP conjugated anti-Flag antibody. A periplasmic fraction of wtCD59 in HB2151 cells is used as a positive control (“pos”).

FIG. 15 shows the specificity and sequence analysis of positive CD59 F2 7-mer binders to GPIIB/IIIa. FIG. 15(A) shows the specificity of 6 positive binders to GPIIb/IIIa from initial panning was further characterized by ELISA. The Immulon 96-well plates were coated with GPIIb/IIIa (specific target) or Mesothelin (non-specific target) or BSA (non-specific target). Periplasmic fractions were isolated and incubated with target or non-target coated 96-well plates. The binding was detected by using an HRP conjugated anti-flag antibody. The data are presented as ELISA signals as measured at an OD of 405 nm. FIG. 15(B) shows the inserted amino acid sequences of the positive binders. Top sequence is the parental sequence with 7-mer insertion. X represents any of the 20 amino acids. The regions covered with grey color are the partial CD59 sequence flanking either end of the 7-mer. C₃-C₁₆: clone names. FIG. 15(C) shows the full length sequences of three of the unique binders obtained from screening the CD59 F2 7-mer library against the integrin GPIIb/IIIa, C3 (SEQ ID NO:111), C10 (SEQ ID NO:112) and C16 (SEQ ID NO:113).

FIG. 16 shows the sequence analysis of positive CD59 F1 9-mer binders to GPIIB/IIIa. The amino acid sequences of the positive binders are shown. The top sequence is the parental sequence with 9-mer insertion. X represents any of the 20 amino acids. The regions covered with grey color are the partial CD59 sequence flanking either end of the 9-mer.

FIG. 17 shows the sequence analysis of positive UPARD3 F2 9-mer binders to GPIIB/IIIa. The amino acid sequences of the positive binders are shown. The top sequence is the parental sequence with 9-mer insertion. X represents any of the 20 amino acids. The regions covered with grey color are the partial UPARD3 sequence flanking either end of the 9-mer.

### DETAILED DESCRIPTION

It is to be understood that this invention is not limited to the particular methodology, protocols, cell lines, animal species or genera, constructs, and reagents described and as such may vary. It is also to be understood that the terminology used herein is for the purpose of describing particular embodiments only, and is not intended to limit the scope of the present invention which will be limited only by the appended claims.

All publications and patents mentioned herein are hereby incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### DEFINITIONS

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

As used herein and in the appended claims, the singular forms “a,” “and,” and “the” include plural reference unless the context clearly dictates otherwise. Thus, for example, reference to “an antibody” is a reference to one or more antibody and includes equivalents thereof known to those skilled in the art, and so forth.

As used herein, the term “three fingered protein domain” or “three fingered domain” or “TFPD” are used interchangeably and refer to a particular protein domain characterized by 3 distinctive β-sheet containing loops or fingers, designated herein as finger 1 (F1), finger 2 (F2) and finger 3 (F3). Typically, a TFPD is about 60-90 amino acids in length and is dominated by three loops or “fingers” forming a large 5 to 6-stranded anti-parallel beta-sheet. Generally, four conserved disulfide bonds are found at the base of the domain from which the three loops extend. A fifth disulfide can be found in some TFPD proteins, for example at tip of the first finger of some human TFPDs. In long chain snake neurotoxins the fifth disulfide is found in the tip of the second loop. The network of 4-5 disulfide bonds imparts structural stability to the TFPD scaffold.

The terms “polypeptide”, “peptide”, “amino acid sequence” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

The single letter abbreviation for a particular amino acid, its corresponding amino acid, and three letter abbreviation are as follows: A, alanine (Ala); C, cysteine (Cys); D, aspartic acid (Asp); E, glutamic acid (Glu); F, phenylalanine (Phe); G, glycine (Gly); H, histidine (His); I, isoleucine (Ile); K, lysine (Lys); L, leucine (Leu); M, methionine (Met); N, asparagine (Asn); P, proline (Pro); Q, glutamine (Gln); R, arginine (Arg); S, serine (Ser); T, threonine (Thr); V, valine (Val); W, tryptophan (Trp); Y, tyrosine (Tyr); and norleucine (Nle).

The term “domain” refers to a stable three-dimensional structure, regardless of size. The tertiary structure of a typical domain is stable in solution and remains the same whether such a member is isolated or covalently fused to other domains. A domain as defined here has a particular tertiary structure formed by the spatial relationships of secondary structure elements, such as beta-sheets, alpha helices, and unstructured loops. In domains of the microprotein family, disulfide bridges are generally the primary elements that determine tertiary structure. In some instances, domains are modules that can confer a specific functional activity, such as avidity (multiple binding sites to the same target), multi-specificity (binding sites for different targets), half-life (using a domain, cyclic peptide or linear peptide) which binds to a serum protein like human serum albumin (HSA) or to IgG (hIgG1, 2, 3 or 4) or to red blood cells.

The “fold” of a polypeptide is largely defined by the linkage pattern of the disulfide bonds (i.e., 1-4,2-6, 3-5). This pattern is a topological constant and is generally not amenable to conversion into another pattern without unlinking and relinking the disulfides such as by reduction and oxidation (redox agents). In general, natural proteins with related sequences adopt the same disulfide bonding patterns. The major determinants are the cysteine distance pattern (CDP) and some fixed non-cys residues, as well as a metal-binding site, if present. In few cases the folding of proteins is also influenced by the surrounding sequences (ie pro-peptides) and in some cases by chemical derivatization (ie gamma-carboxylation) of residues that allow the protein to bind divalent metal ions (ie Ca++) which assists their folding. For the vast majority of polypeptides such folding help is not required.

However, proteins with the same bonding pattern may still comprise multiple folds, based on differences in the length and composition of the loops that are large enough to give the protein a rather different structure. Determinants of a protein fold are any attributes that greatly alter structure relative to a different fold, such as the number and bonding pattern of the cysteines, the spacing of the cysteines, differences in the sequence motifs of the inter-cysteine loops (especially fixed loop residues which are likely to be needed for folding, or in the location or composition of the calcium (or other metal or co-factor) binding site.

The term “scaffold” refers to a polypeptide platform for the engineering of new products, e.g. proteins or antibody mimetics, with tailored functions and characteristics. Such scaffolds are useful for the construction of protein libraries. A scaffold is typically defined by the conserved residues that are observed in an alignment of a family of sequence-related proteins. Fixed residues may be required for folding or structure, especially if the functions of the aligned proteins are different. Thus, when designing proteins from the scaffold, amino acid residues that are important for the framework's favorable properties are retained, while others residues may be randomized or mutated. A full description of a protein scaffold may include the number, position or spacing and bonding pattern of the cysteines, as well as position and identity of any fixed residues in the loops.

By “randomized” or “mutated” is meant including one or more amino acid modifications relative to a template sequence. By “randomizing” or “mutating” is meant the process of introducing, into a sequence, such an amino acid modification. Randomization or mutation may be accomplished through intentional, blind, or spontaneous sequence variation, generally of a nucleic acid coding sequence, and may occur by any technique, for example, PCR, error-prone PCR, or chemical DNA synthesis. By a “corresponding, non-mutated protein” is meant a protein that is identical in sequence, except for the introduced-amino acid mutations. The amino acid may be modified by a substitution, i.e. a replacement of one amino acid for a different amino acid. The amino acid may also be modified by an insertion or deletion of amino acid residues.

The term “antibody mimetic” or “mimetic” as used herein is meant a protein that exhibits binding similar to an antibody but is a smaller alternative antibody or a non-antibody protein.

As used herein, by “non-antibody protein” is meant a protein that is not produced by the B cells of a mammal either naturally or following immunization of a mammal.

“Non-naturally occurring” as applied to a protein means that the protein contains at least one amino acid that is different from the corresponding wild-type or native protein. Non-natural sequences can be determined by performing BLAST search using, e.g., the lowest smallest sum probability where the comparison window is the length of the sequence of interest (the queried) and when compared to the non-redundant (“nr”) database of Genbank using BLAST 2.0. The BLAST 2.0 algorithm, which is described in Altschul et al. (1990) J. Mol. Biol. 215:403-410, respectively. Software for performing BLAST analyses is publicly available through the National Center for Biotechnology Information.

As used herein, the term “isolated” means separated from constituents, cellular and otherwise, in which the polynucleotide, peptide, polypeptide, protein, antibody, or fragments thereof, are normally associated with in nature.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Recombinant” as applied to a polynucleotide means that the polynucleotide is the product of various combinations of cloning, restriction and/or ligation steps, and other procedures that result in a construct that is distinct from a polynucleotide found in nature.

A “vector” or “plasmid” is a nucleic acid molecule, preferably self-replicating, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector described herein. An example of a host cell described herein are CHO K1 cells.

A “pharmaceutical composition” is intended to include the combination of an active agent with a pharmaceutically acceptable carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON′S PHARM. SCl., 15th Ed. (Mack Publ. Co., Easton (1975)).

### Three Fingered Protein Domain

Antibody mimetics represent a novel class of biologics derived from non-traditional antibody-based protein domains or scaffolds. The structural features of a robust mimetic include intrinsic overall conformational stability and the ability to tolerate extensive modification to variable loop regions within the scaffold. Such features allow the generation of large libraries of products having novel binding characteristics.

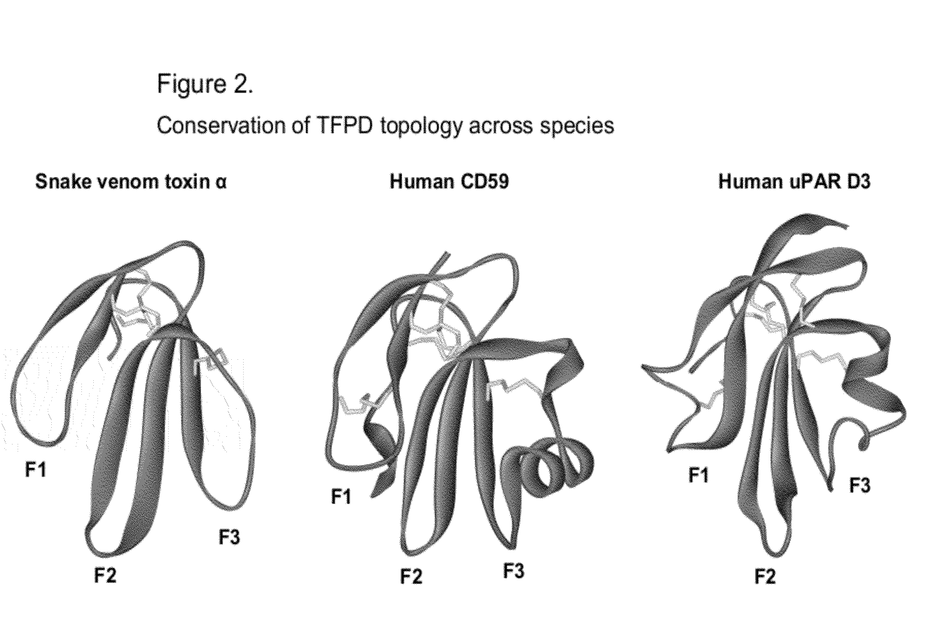

The three fingered protein domain (TFPD) was identified as a novel antibody mimetic through a systematic search of the public protein databases with specific structural and functional criteria that coincided with the desired profile for a novel and robust mimetic. The TFPD is small having about 60 to 90 amino acid residues, disulfide-rich comprising about 4 or 5 disulfide bonds, and contains three distinctive β-sheet containing loops or “fingers” as depicted in FIG. 2. Three finger protein domains are present throughout vertebrates and exhibit broad functional diversity, from potent and lethal snake venoms (e.g α-bungarotoxin) to physiologically important cell surface receptors (e.g. urokinase receptor).

The human TFPD was identified as a potential mimetics scaffold using search criteria that matched the profile for a well-behaved mimetic. The criteria selected for protein domains that were small (50-100 amino acids in length), stable (soluble, extracellular), and human in origin. In addition structures were sought that would be amenable to the generation and display of libraries for screening novel targets, particularly integral membrane proteins, such as GPCRs and ion channels. Thus, protein domains with loops that could be varied in composition and extended in length could be of great value in binding pockets or deep crevices found in certain target proteins. It has also been shown that the extended CDR3 loops found in antibodies derived from camelids and sharks also display this feature. The extended CDR3 loop present in the variable heavy chain of a shark IgNAR specific for apical membrane antigen (AMA1) from plasmodium has been shown to penetrate the deep hydrophobic cleft of the protein.

The TFPD contains three loop regions, or “fingers,” and mutagenesis studies have shown that the contact residues for several of the snake toxins involved in binding target proteins such as the acetylcholine receptor reside within the loops. The library design strategy takes advantage of the natural binding properties of TFPDs by building diversity within loop regions of the scaffold. In doing so it is believed that TFPD binders can be developed against proteins that have proven difficult to target in the past, such as integral membrane proteins (e.g. G-protein coupled receptors and ion channels).

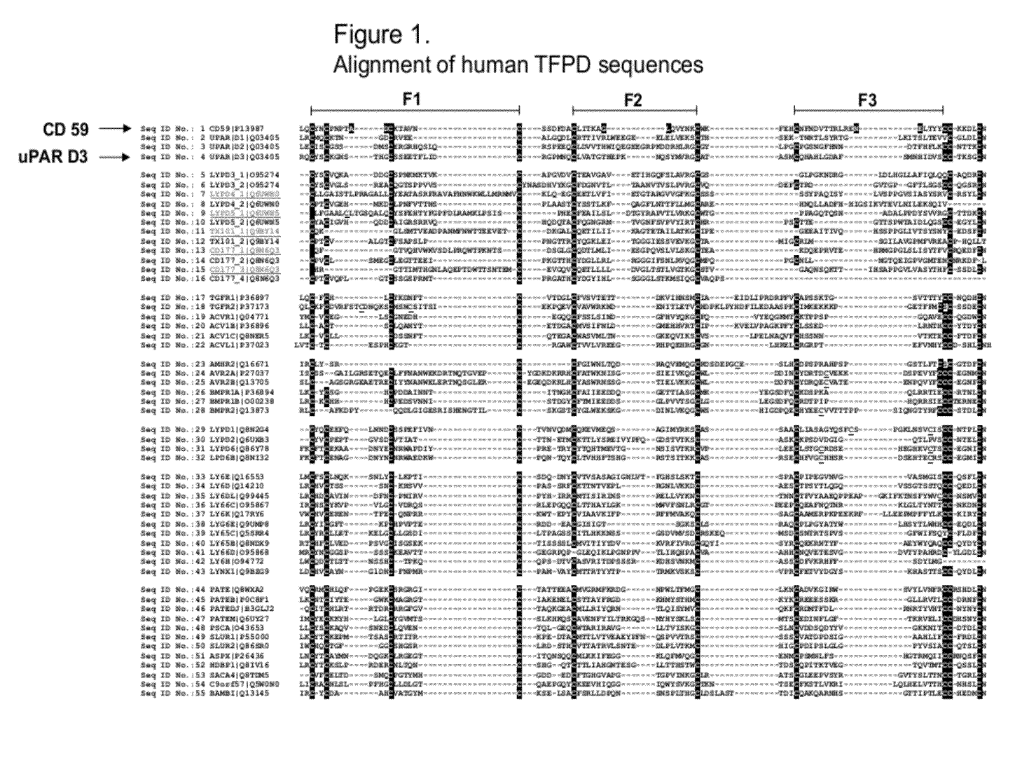

TFPDs that were identified as potential mimetic scaffolds include, but are not limited to, those listed in FIG. 1. Such TFPDs include genes within the Ly-6/uPAR (LU) protein domain family, for example CD59 (SwissProt/UniProt ID# P13987), urokinase receptor (uPAR) domain 1 (Q03405), uPAR domain 2 (Q03405), and uPAR domain 3 (Q03405). The TFPDs also include genes within the Transforming Growth Factor-β receptor (TGFR) family such as TGFR domain 1 (P36897), TGFR domain 2 (P37173), ACVR1 (Q04771), ACV1B (P36896), ACV1C (Q8NER5), ACVL1 (P37023), AMHR2 (Q16671), AVR2A (P27037), AVR2B (Q13705), BMR1B (P36894), BMR1A (O00238), and BMPR2 (Q13873). Other TFPDs include members of the human uPAR gene cluster such as LYPD3-1 (O95274), LYPD3-2 (O95274), LYPD4-1 (Q6UWN0), LYPD4-2 (Q6UWN0), LYPD5-1 (Q6UWN5), LYPD5-2 (Q6UWN5), TX101-1 (Q9BY14), TX101-2 (Q9BY14), CD177-1 (Q8N6Q3), CD177-2 (Q8N6Q3), CD177-3 (Q8N6Q3), and CD177-4 (Q8N6Q3). Additionally, TFPDs include human genes of the LU family such as LYPD1 (Q8N2G4), LYPD2 (Q6UXB3), LYPD6 (Q86Y78), LPD6B (Q3NI32), LY6E (Q16553), LY6D (Q14210), LY6DL (Q99445), LY66C (O95867), LY6K (Q17RY6), LYG6E (Q9UMP8), LY65C (Q6SRR4), LY65B (Q8NDX9), LY66D (O95868), LY6H (O94772), LYNX1 (Q9BZG9), PATE (Q8WXA2), PATEB (POC8F1), PATEDJ (B3GLJ2), PATEM (Q6UY27), PSCA (O43653), SLUR1 (P55000), SLUR2 (Q86SR0), ASPX (P26436), HDBP1 (Q81V16), SACA4 (Q8TDM5), C9orf57 (Q5W0N0), and BAMBI (Q13145).

In addition, inactive mutants of the human TFPDs can also serve as potential mimetic scaffolds. Such inactive mutants are useful as potential scaffolds because they allow introduction of novel binding characteristics with neutral function while still retaining overall conformational stability.

For CD59, mutations at position 24 or 40 are known to generate inactive mutants. Described in Huang Y et al., J. Biol. Chem. (2005) 280:34073-79, it was shown with an alanine scan of the human CD59 protein that both the Asp24Ala and the Trp40Ala CD59 mutants, when expressed in CHO cells, are nearly 100% inactive in their ability to inhibit complement lysis of the CHO cells as compared to CD59 wild type expressing CHO cells. Additionally, in Bodian D L et al., J. Exp. Med. (1997) 185:507-16, mutational analysis of human CD59 was carried out and the mutants were characterized for functional activity and the ability to be recognized by active conformation-specific CD59 antibodies. This analysis demonstrated that the CD59 mutant, Trp40Glu, is inactive, but remained properly folded as determined by a conformation specific antibody. The activity of the hCD59 mutant, Asp24Arg, was also shown to be disrupted yet still folded properly by the conformationally sensitive CD59 antibodies. Thus, inactive CD59 mutants can be used as template scaffolds for display, library generation, and screening. In some embodiments, the inactive CD59 mutant has a modification at position Asp24 and Trp40, individually and in combination. In some embodiments, the inactive CD59 mutants may comprise the modification Asp24Ala, Asp24Arg, Trp40Ala, Trp40Glu, or combinations of thereof.

Further, for the uPAR domain 3, it has been shown that a specific 9-mer peptide corresponding to uPAR 240-248 blocks integrin binding, and further that a single amino acid mutant at position 245, uPAR Ser245Ala, completely abrogates integrin binding and signaling. Thus, in some embodiments, an inactive mutant of uPAR domain 3 can be used as a template scaffold for display, library generation, and screening. In some embodiments, the inactive mutant of uPAR domain 3 comprises a modification at position Ser245, and in some embodiments, the modification comprises Ser245Ala.

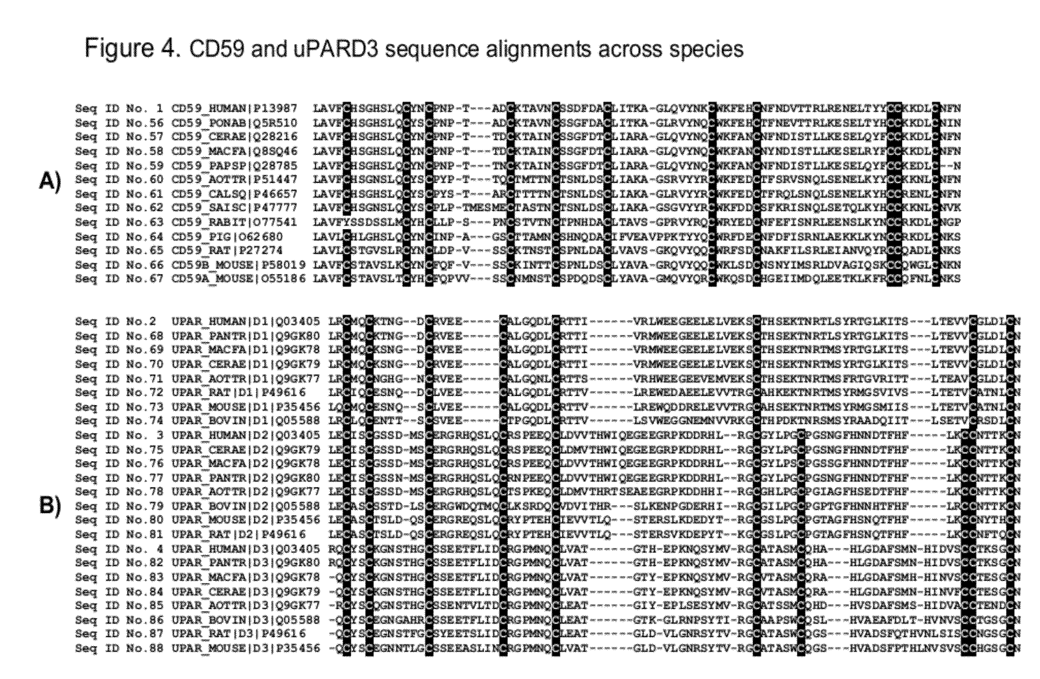

It is also contemplated that TFPDs from other species can be used as template scaffolds for display, library generation and screening. FIG. 4A shows an alignment of CD59 from HUMAN, human; AOTTR, owl monkey; CALSQ, marmoset; CERAE, African green monkey; MACFA, crab-eating macaque; PAPSP, baboon; PONAB, orangutan; SAISC, squirrel monkey; PANTR, chimpanzee; RABIT, rabbit; PIG, pig; RAT, rat; and MOUSE, mouse. These species display conserved disulfides and therefore should retain similar tertiary structure. Similarly, FIG. 4B shows an alignment of uPAR domains 1-3 from several species also displaying the conserved disulfides.

Accordingly, one aspect of the application is a polypeptide comprising a three finger protein domain (TFPD) wherein said TFPD has an amino acid sequence that has been modified relative to the amino acid sequence of a naturally occurring TFPD such that the modified TFPD binds to a specified target molecule. In some embodiments, the modification is at a position in finger 1 (F1) of said TFPD, finger 2 (F2) of said TFPD, finger 3 (F3) of said TFPD, or a combination of two or more fingers.

In some embodiments, the specified target molecule bound by the modified TFPD is not bound by the naturally occurring TFPD. In some embodiments, the specified target molecule includes, but is not limited to, a protein, a nucleotide, an antibody, a small molecule or other antigen of interest. In some embodiments, the specified target molecule is a therapeutic target. By binding a therapeutic target, the modified TFPD could function as an agonist or antagonist of the target protein. Thus, the modified TFPD is potentially useful as a pharmaceutical compound for therapeutic or diagnostic purposes.

Modifications may be made by substituting one amino acid residue for a different amino acid residue, for example by mutation, directed evolution, or other suitable method. Single or multiple specific amino acids may be selected for substitution or alternatively random substitutions may be generated. In some embodiments, the polypeptide comprises a specific predetermined substitution. In other embodiments, the polypeptide comprises a random substitution of any amino acid residue.

Modifications may also be made by inserting a sequence of amino acid residues. Insertions may be as small as 1 residue. Alternatively, insertions can be of any length. For example, insertions of 7 residues, 9 residues, and 11 residues as described in the Examples below may be inserted into F1, F2 or F3. The insertions can also be made between any residue along each finger. Further, an insertion may involve the substitution of one or more of the existing residues in conjunction with the addition of one or more amino acid residues. For example, two amino acid residues may be substituted with seven amino acid residues, thereby giving a net insertion of 5 amino acid residues.

In some embodiments, modifications may also be made by deletion. Such deletions would have to be done with prudence to prevent drastic alteration of the three-dimensional structure.

In some embodiments, modifications may be made in a combination of two or more fingers. For example modifications may be made in F1 and F2, F2 and F3, F1 and F3, or F1, F2 and F3. Additionally, modifications may be either substitutions or insertions or combinations thereof. For example, a substitution may be made in F1 and an insertion in F3.

Another aspect of the application provides libraries of polypeptides comprising a plurality of three finger protein domains (TFPD) wherein said TFPD have amino acid sequences that has been modified relative to the amino acid sequences of corresponding naturally occurring TFPD such that the modified TFPD binds to a specified target molecule. Such libraries may include random modifications generated in a single finger, for example F2, or may include random modifications generated in multiple fingers.

Also provided are polynucleotides encoding for the polypeptides described herein, vectors containing the polynucleotides, and host cells containing these vectors.

### Multi-Specific Scaffolds

Bispecific antibodies represent an alternative antibody format that can target and engage two different target molecules simultaneously with a single molecule. The concept of multivalent antibody mimetics can be extended to the addition of effector functions to the scaffold. For example, bispecific mimetics have been designed in which one domain binds to human serum albumin, thereby extending the half-life of the mimetic, while the other domain binds to a target molecule.

In another embodiment, the TFPD can also be engineered with ligands involved in tumorigenesis. For example, the TFPD can be engineered with one binding domain targeted to CD3 on T-cells and another domain targeted to a cancer-specific cell surface antigen. The anti-CD3 domain creates an immunological synapse between a T cell and a tumor cell, ultimately inducing a self-destruction process in the tumor cell referred to as apoptosis, or programmed cell death.

In some embodiments, the TFPD scaffolds may further comprises an element imparting effector function. For example, effector functions would include TFPD binders that prolong TFPD half-life in the circulation that specifically bind to human serum albumin (HSA). In another example, the TFPD may comprise an effector function such that it can be chemically conjugated to a polyethylene glycol (PEG) molecule or hydroxyethyl starch (HES) molecule to prolong TFPD half-life in circulation. In another example, the TFPD is fused to the Fc region of an immunoglobulin to enhance receptor (Fc)R) mediated effector functions, such as antibody-dependent cell-mediated cytotoxicity (ADCC) and antibody-dependent cell-mediated phagocytosis (ADCP). In another example, a tumor specific TFPD is chemically conjugated with a cell killing toxophore that would enable tumor specific TFPD targeting and cytotoxicity.

In another aspect of the application, bispecific or multivalent TFPDs can be generated within a single domain such that the individual fingers or loops bind different targets. For example, a single TFPD can be generated such that the F1 loop binds a particular target protein, while the F2 loop binds a different target protein.

Alternatively, TFPD multimers can be designed and expressed as fusion proteins in which each TFPD monomer binds a different target. For example, a TFPD dimer could be generated in which each TFPD monomer binds to and blocks the function of two different receptors overexpressed on tumor cells. In another example, a TFPD dimer with an extended half-life can be generated in which one TFPD monomer binds human serum albumin with high affinity, while the other TFPD monomer binds a target receptor overexpressed on tumor cells.

Evidence for TFPD multimers in nature comes from the several TFPDs in mammals that are expressed as ectodomains of cell surface receptors, for example the urokinase receptor (uPAR) and the members of the TGF-β receptor family. In the case of uPAR it has been shown that the three TFPDs making up the extracellular region of uPAR have different binding properties, with domains 1 and 2 primarily involved in binding the uPAR ligand, urokinase, while domain 3 is involved in binding the integrin α5β1.

### Use

The TFPD scaffolds described herein are useful in generating libraries of proteins having novel binding characteristics.

In some embodiments, the TFPD scaffolds are useful in generating antibody mimetics. Such antibody mimetics may be evolved to bind any target antigen of interest. These proteins have properties superior to those of natural antibodies and can be evolved rapidly in vitro. Accordingly, these antibody mimics may be employed in place of antibodies in all areas in which antibodies are used, including in the research, therapeutic, and diagnostic fields. In addition, because these scaffolds possess solubility and stability properties superior to antibodies, the antibody mimetics described herein may also be used under conditions which would destroy or inactivate antibody molecules. Finally, because the scaffolds allow binding to virtually any compound, these molecules provide completely novel binding proteins which also find use in the research, diagnostic, and therapeutic areas.

In another embodiment, the target of binding can be carried out in solution. For example, the technique was described by Viti F et al. Methods in enzymology (2000) vol. 326. p. 480-505; by Huang L et al. J. of Mol. Recognit. (2005) 18: 327-333. The phages carrying the antibody mimetic scaffolds bind to biotinylated target in solution, and the complex is then captured by using streptavidin coupled Dynabeads. The target of binding can also be carried out on the cell surface. For example, a method has been described to select antibodies to cell-surface antigens using magnetic sorting techniques (Antibody phage display: methods and protocols by Philippe M. O'Brien, Robert Aitken, Chapter 18, p219-226). Eisenhardt S U et al. (2007, Nature Protocols vol 2. 3063-3073) also described a protocol that allows the selection of highly specific scFv antibody clones that are able to discriminate between various conformational states of cell surface receptors (targets).

In one particular example, the TFPD scaffold may be used as the selection target. For example, if a protein is required that binds a specific peptide sequence presented in a ten residue loop, a single TFPD clone is constructed in which one of its loops has been set to the length of ten and to the desired sequence. The new clone is expressed in bacteria and purified, and then immobilized on a solid support. A phage display library based on an appropriate scaffold is then allowed to interact with the support, which is then washed, and desired molecules eluted and re-selected as described above.

Similarly, the scaffolds described herein, for example, the TFPD scaffold, may be used to find natural proteins that interact with the peptide sequence displayed by the scaffold, for example, in a TFPD finger. The scaffold protein, such as the TFPD protein, is immobilized as described above, and a phage display library is screened for binders to the displayed loop. The binders are enriched through multiple rounds of selection and identified by DNA sequencing.

### Directed Evolution of Scaffold-Based Binding Proteins

The antibody mimetics described herein may be used in any technique for evolving new or improved binding proteins. In one particular example, the target of binding is immobilized on a solid support, such as a column resin or microtiter plate well, and the target contacted with a library of candidate scaffold-based binding proteins. Such a library may consist of antibody mimetic clones, such as TFPD clones constructed from the wild type TFPD scaffold through randomization of the sequence and/or the length of the TFPD fingers. If desired, this library may consist of a fusion protein library displayed on filamentous phage as described in G P Smith, Science (1985), J McCafferty et al., Nature (1990), or H B Lowman et al., Biochemistry (1991). The library may also be displayed on the surface of yeast [see E T Boder et al., Nat. Biotech. (1997)] or mammalian cells (see R R Beerli et al. Proc. Nat. Acad. Sci. USA (2008)], or in vitro using ribosomal display [see C Zahnd et al., Nat. Methods (2007)]. In another example, the library may be an RNA-protein fusion library generated, for example, by the techniques described in Szostak et al., U.S. Ser. No. 09/007,005 and 09/247,190; Szostak et al., WO98/31700; and Roberts & Szostak, Proc. Natl. Acad. Sci. USA (1997) vol. 94, p. 12297-12302. Alternatively, it may be a DNA-protein library (for example, as described in Lohse, DNA-Protein Fusions and Uses Thereof, U.S. Ser. No. 60/110,549, U.S. Ser. No. 09/459,190, and WO 00/32823). The fusion library is incubated with the immobilized target, the support is washed to remove non-specific binders, and the tightest binders are eluted under very stringent conditions and subjected to PCR to recover the sequence information or to create a new library of binders which may be used to repeat the selection process, with or without further mutagenesis of the sequence. A number of rounds of selection may be performed until binders of sufficient affinity for the antigen are obtained.

### Target Protein Capture and Detection

Selected populations of TFPD scaffold-binders may be used for detection and/or quantitation of analyte targets, for example, in samples such as biological samples. To carry out this type of diagnostic assay, selected scaffold-binders to targets of interest are immobilized on an appropriate support to form multi-featured protein chips. Next, a sample is applied to the chip, and the components of the sample that associate with the binders are identified based on the target-specificity of the immobilized binders. Using this technique, one or more components may be simultaneously identified or quantitated in a sample (for example, as a means to carry out sample profiling).

Methods for target detection allow measuring the levels of bound protein targets and include, without limitation, radiography, fluorescence scanning, mass spectroscopy (MS), and surface plasmon resonance (SPR). Autoradiography using a phosphorimager system (Molecular Dynamics, Sunnyvale, Calif.) can be used for detection and quantification of target protein which has been radioactively labeled, e.g., using 35S methionine. Fluorescence scanning using a laser scanner (see below) may be used for detection and quantification of fluorescently labeled targets. Alternatively, fluorescence scanning may be used for the detection of fluorescently labeled ligands which themselves bind to the target protein (e.g., fluorescently labeled target-specific antibodies or fluorescently labeled streptavidin binding to target-biotin, as described below).

Mass spectroscopy can be used to detect and identify bound targets based on their molecular mass. Desorption of bound target protein can be achieved with laser assistance directly from the chip surface as described below. Mass detection also allows determinations, based on molecular mass, of target modifications including post-translational modifications like phosophorylation or glycosylation. Surface plasmon resonance can be used for quantification of bound protein targets where the scaffold-binder(s) are immobilized on a suitable gold-surface (for example, as obtained from Biacore, Sweden).

### EXAMPLES

It will be apparent to those skilled in the art that the examples and embodiments described herein are by way of illustration and not of limitation, and that other examples may be used without departing from the spirit and scope of the present invention, as set forth in the claims.

### Example 1

### Identification of the Human TFPD Scaffold from Database Mining

Several criteria were employed in searching the public protein databases to identify novel protein folds that might serve as good scaffolds for an antibody mimetic. Ideally the scaffold is small, between 50 and 100 amino acids in length, of human origin to minimize immunogenicity, soluble and extracellular to ensure ease of handling, and have a known three-dimensional structure. In addition there should be data demonstrating that a recombinant form of the protein domain can be expressed in high levels in a heterologous host organism, such as E. coli or Pichia. In addition it is highly desirable to have mutagenesis or protein engineering data showing that the scaffold is amenable to modifications that would include introduction of novel binding properties.

Potential scaffolds were also selected with the ability to bind targets that have proven difficult in the past with traditional antibody-based approaches, particularly integral membrane proteins, such as GPCRs and ion channels. This structural and functional adaptability of the scaffold is important in demonstrating that the scaffold can be re-engineered to bind diverse target classes.

Using the criteria outlined above 88 protein domains were identified from 752 domains in the SMART (Simple Modular Architecture Research Tool, 5.1 release) database. After mapping these domains to the Structural Classification of Proteins (SCOP) database and blasting for the most highly conserved sequences across species 17 protein domains were selected, and the three fingered protein domain [SM001526, Ly-6/uPA receptor-like domain (LU)] was chosen for experimental validation as a mimetics scaffold.

Within the SCOP database (Structural Classification of Proteins) the “snake toxin-like” fold [accession #57301] consists of 36 protein structures within three superfamilies (snake venom toxins, dendroaspin, and extracellular domain of cell surface receptors). The “snake toxin-like” fold is defined as 60-75 amino acids in length, with two beta sheets encompassing three large loops or “fingers”. A network of 4-5 disulfides imparts structural stability to the TFPD scaffold.

The human TFPD scaffold is related to the murine Ly-6 antigen and encompasses at least 53 known human proteins that occur primarily within the ecto-domains of cell surface receptors (e.g. the TGF-beta receptor family, urokinase receptor, uPAR), as well as GPI-linked proteins (e.g. CD59, PSCA). FIG. 1 lists the 53 known human TFPD sequences with their accession numbers from UniProt (http://www.uniprot.org/). The two human TFPD domains, CD59 and the third domain in the human urokinase receptor (uPARD3), were selected as display candidates, based upon the large amount of structural and functional data known about these protein domains, including mutagenesis data describing inactive mutants, as well as structural data on complexes with their respective ligands.

The TFPDs share a very similar topology across species (FIG. 2). The fold is distinguished by a structural core of 4 disulfides from which emanate the three loops or “fingers”. A fifth disulfide is present in finger 1 in the human TFPDs that may provide additional structurally stability to the distal portion of this loop.

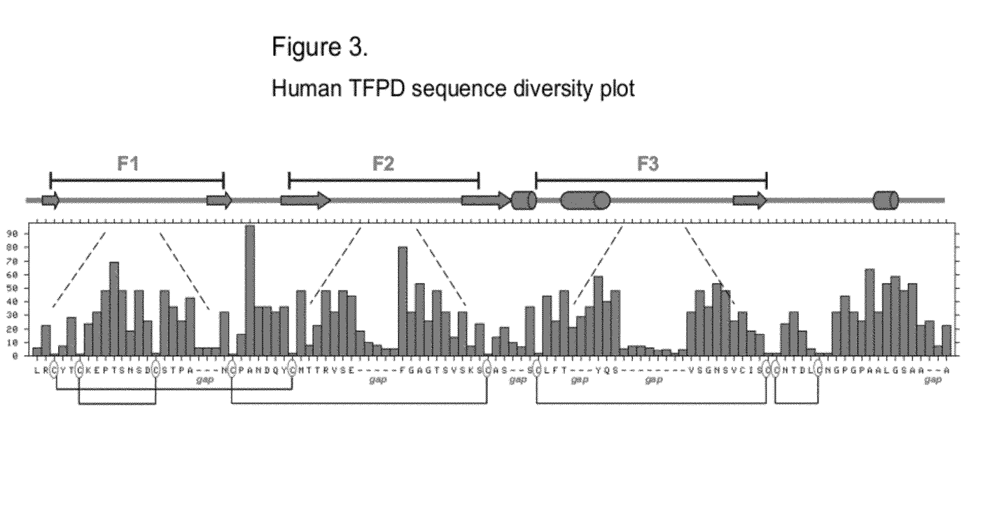

Even though TFPDs share a remarkably similar topology within the human TFPDs there exists a large sequence diversity, particularly within the three finger regions. FIG. 3 shows a sequence diversity plot for 17 human TFPDs from the SCOP database. The high degree of sequence variation reflects the functional diversity of the domain as well as the ability of the TFPD scaffold to accommodate a high degree of modification, while retaining the overall conformation. The large gap regions tend to occur within the loops indicating that the fingers can tolerate variable lengths in addition to diversity in composition.

The validation of the TFPD as a mimetics scaffold involved three stages. First, each of fingers or loops was tested for its ability to accommodate additional sequence within the tip regions by inserting 7, 9, or 11 residues from the RGD containing protein, eristostatin. Using human CD59 it was that demonstrated specific integrin binding activity for each of the insertion variants while retaining native binding to the C9 complement protein. In addition, specific integrin binding was also shown for the F2 loop of human uPAR domain 3. Second, it was demonstrated that the hCD59 wild type and the insertion variants could be displayed on phage as gIII fusion proteins, and that they maintained binding functionality. Third, a highly diverse random library was constructed within the tip of loop 2 of hCD59 and used to screen a soluble target protein, GPIIb/IIIa.

### Example 2

### Materials and Methods

### Plasmid Construction

All transgenes were cloned into a pM197 phagemid vector. This vector was derived from pCANTAB5E (Gene bank #U14321), in which the gill signal sequence was replaced with the pelB leader sequence and a FLAG-tag was inserted in front of the E-tag. FLAG or E-tag was used for the purposes of purification and detection.

The human CD59 gene encodes a 77 amino acid mature peptide. The human mature CD59 sequence (Gene bank#NM-203330) was codon-optimized for bacterial expression.

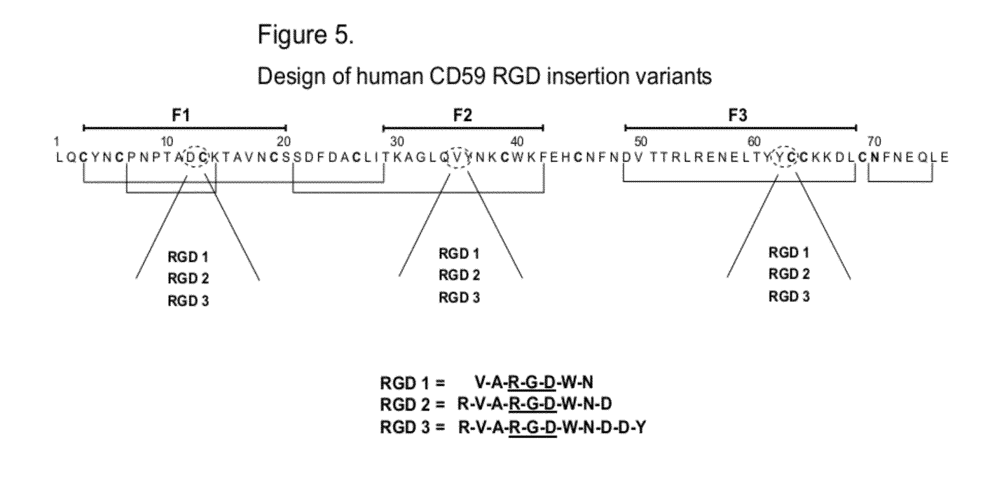

Three CD59/F2/RGD variants were generated. Three peptides from the RGD loop of eristostatin comprising 7 to 11 residues were inserted into finger 2 (F2) of CD59 and replaced residues Gly32-Leu33 (insertion site), see FIG. 5. The RGD sequences are VARGDWN (RGD1, SEQ ID NO:89), RVARGDWND (RGD2, SEQ ID NO:90), and RVARGDWNDDY (RGD3, SEQ ID NO:91). The codon-optimized wild-type CD59 and three CD59/F2/RGD variants were synthesized through BlueHeron (Invitrogen). These genes were flanked by SfiI and NotI and cloned into pM197 to create pGT2042, pGT2043 and pGT2044.

The same principle was applied to finger 1 and finger 3 of CD59. A11D12 or N57E58 were deleted and replaced with the three RGDsequences. Three CD59/F1/RGD and three CD59/F3/RGD variants were also generated. To make CD59/F1/RGD variants, three pairs of oligos were synthesized: GER283 (5′-CGGCCATGGCTCTTCAATGCTA CAACTGTCCTAACCCGACTGTGGCACGTGGTGATTGGAATTGTAAAACCGC-3′, SEQ ID NO:92) and GER284(5′-GGTTTTACAATTCCAATCACCACGTGCCACAGTCGGGTTAGGACAGTTGTAGCATTGAAG AGCCATGGCCGGCT-3′, SEQ ID NO:93) for RGD1; GER285 (5′-CGGCCATGGCTCTTCAATGCTACAACTGTCCTAACCCGACTCGCGTGGCACGTGGTGATT GGAATGACTGTAAAACCGC-3′, SEQ ID NO:94) and GER286 (5′-GGTTTTACAGTCATTCCAATCACCA CGTGCCACGCGAGTCGGGTTAGGACAGTTGTAGCATTGAAGAGCCATGGCCGGCT-3′, SEQ ID NO:95) for RGD2; GER287 (5′-CGGCCCTTCAATGCTACAACTGTCCTAACCCGACTCGCGTGGCACGTGGTGATTGGAATG ACGACTACTGTAAAACCGC-3′, SEQ ID NO:96) and GER 288 (5′-GGTTTTACAGTAGTCGTCATTCCAATCACCACGTGCCACGCGAGTCGGGTTAGGACAGTT GTAGCATTGAAGGGCCGGCT-3′, SEQ ID NO:97) for RGD3. The corresponding pair of oligos was annealed so that the double strand fragments were flanked by SfiI and SacII, and then cloned into pM197 to create pGT2046, pGT2047 and pGT2048. To make CD59/F3/RGD variants, three pairs of oligos were also synthesized: GER289 (5′-CGCGTCTCCGTGAAGTGGCACGTGGT GATTGGAATCTTAC-3′, SEQ ID NO:98) and GER290 (5′-GTAAGATTCCAATCACCACGTGCCACTTCACGGAGA-3′, SEQ ID NO:99) for RGD1; GER291 (5′-CGCGTCTCCGTGAACGCGTGGCACGTGGTGATTGGAATGACCTTAC-3′, SEQ ID NO:100) and GER292 (5′-GTAAGGTCATTCCAATCACCACGTGCCACGCGTTCACGGAGA-3′, SEQ ID NO:101) for RGD2; GER293 (5′-CGCGTCTCCGTGAACGCGTGGCACGTGGTGATTGGAATGACGACTACCTTAC-3′, SEQ ID NO:102) and GER294 (5′-GTAAGGTAGTCGTCATTCCAATCACCACGTGCCACGCGTTCACGGAGA-3′, SEQ ID NO:103) for RGD3. The corresponding pair of oligos was annealed so that the double strand fragments were flanked by MluI and SnaBI, and then cloned into pM197 to create pGT2049, pGT2050 and pGT2051.

Human uPAR domain 3 (gene bank #X51675, contains amino acids 192 to 283 of mature protein) was codon-optimized for bacterial expression and synthesized through BlueHeron (Invitrogen). The gene was flanked by SfiI and NotI and cloned into pM197 to create pGT2036.

To make uPAR D3/F2/RGD variants, three pairs of oligos were synthesized: GER297:(5′-CCGGTACTCACGAAGTGGCACGTGGTGATTGGAATAATCAATCTTATATGGTCCGC-3′, SEQ ID NO:104) and GER298:(5′-GGACCATATAAGATTGATTATTCCAATCACCACGTGCCACTTCGTGAGTA-3′, SEQ ID NO:105) for RGD1; GER299:(5′-CCGGTACTCACGAACGCGTGGCACGTGGTGATTGGAATGACAATCAATCTTATATGGTCC GC-3′, SEQ ID NO:106) and GER300:(5′-GGACCATATAAGATTGATTGTCATTCCAATCACCACGTGCCACGCGTTCGTGAGTA-3′, SEQ ID NO:107) for RGD2; GER301:(5′-CCGGTACTCACGAACGCGTGGCACGTGGTGATTGGAATGACGACTACAATCAATCTTATA TGGTCCGC-3′, SEQ ID NO:108) and GER302:(5′-GGACCATATAAGATTGATTGTAGTCGTCATTCCAATCACCACGTGCCACGCGTTCGTGAG TA-3′, SEQ ID NO:109) for RGD3.

The corresponding pair of oligos was annealed so that the double strand fragments were flanked by AgeI and SacII, and then cloned into pM197 to create pGT2053, pGT2054, and pGT2055.

### Bacterial Expression and Periplasmic Extraction

Single bacterial clone (from HB2151 cells) was inoculated in 2 ml LB medium with 100 μg/ml ampicillin overnight at 37° C. The preculture was diluted 1:100 in fresh LB medium with 100 μg/ml ampicillin and shaked at 30° C. to give OD₆₀₀=0.5. The cells were then induced by the addition of IPTG (final concentration 1 mM) and grown at 30° C. for another 3 to 4 hours before harvesting for periplasmic fraction extraction. The periplasmic extraction procedure was performed using PeriPreps periplasting kit (Epicentre Biotechnologies, Wisconsin) according to the manufacturer's instruction.

### Western Blotting

The bacterial lysates or recombinant phage was loaded and separated on 4-12% Bis-Tris gel (Invitrogen, Carlsbad, Calif.). The gel was blotted onto Nitrocellulose membrane through iBlot (Invitrogen, Carlsbad, Calif.) and immediately blocked in 0.5% milk PBS. The membrane was then incubated with HPR conjugated anti-Flag antibody (Sigma, St. Louis, Mo., 1:3000 in 0.05% PBST) for 10 minutes using iSNAP device (Bio-Rad, Hercules, Calif.). The bands were detected using ECL Plus (Amersham Biosciences, Pittsburgh, Pa.).

### C9 Binding Assay

Immulon 4 HBX plates were coated with 50 ng/50 μl per well C9 protein (from Quidel, Calif.) in bicarbonate buffer overnight at 4° C. The plates were blocked 1 hour with 200 μl/well 3% BSA in PBS while shaking Periplasmic samples or phage were prepared as 50 μl/well within desired percentage of PBS and incubated 2 hours at RT. The plates were then washed 4 times with 0.05% PBST (0.05% Tween 20 in PBS buffer) washing buffer using a plate-washer (Bio-Tek). 50 μl/well of HRP-conjugated anti-E-tag antibody (GE, 1:5000 dilution) or anti-M13 antibody (NEB, 1:2500 dilution) was incubated at RT for 1 hour following four times washing with PBST (0.05% Tween 20). Absorbance at 405 nm was measured after 100 μl/well of ABTS (Sigma) addition.

### GPIIb/IIIa Binding and Competitive Assay

Immulon 1B plates were coated with 200 ng/100 μl per well GPIIb/IIIa protein (Innovative research Inc,) in coating buffer (20 mM Tris, pH 7.5, 150 mM NaCl, 1 mM each of CaCl₂, MgCl₂, MnCl₂) overnight at 4° C. The plates were blocked 1 hour with 200 μl/well of blocking buffer comprising 3% BSA in binding buffer (50 mM Tris pH 7.5, 100 mM NaCl, 1 mM each of CaCl₂, MgCl₂, MnCl₂) while shaking. For direct binding assay, periplasmic samples or phage were prepared (50 μl/well) within a desired percentage of binding buffer and incubated 2 hours at RT. In the case of competitive assay, a serial diluted (1:10 dilution stepwise) competitors RGD peptide BHRF1, BHRF1-KG (BHRF1 linked to KG (factor VIII)) and fibrinogen, negative control KG were mixed with periplasmic fraction or phage and then added to GPIIb/IIIa-coated 96-well plate followed by 2 hours of room temperature incubation. The plates were then washed 4 times with BB using a plate-washer (Bio-Tek). 50 μl/well of HRP-conjugated anti-E-tag antibody (GE, 1:5000 dilution) or anti-M13 antibody (NEB, 1:2500 dilution) was incubated at RT for 1 hour following four times washing with binding buffer. Absorbance at 405 nm was measured after 100 μl/well of ABTS (Sigma) addition.

### Library Construction and Cloning

A random library was generated by modifying the F2 domain of CD59. The residues Gly32 and Leu33 were deleted and replaced with a 7 amino acid residue insertion in which each position is randomized with all 20 possible amino acids using triphosphoroamidite-based synthesis, thus eliminating frame shifts, stop codons and overrepresentation of some amino acids from the library.

The direct strand primers containing the library were synthesized from Gene-Link Inc. (Hawthorne, N.Y.): 5′-TCGATGCATGCTTAATTACTAAAGCCXXXXXXXCAGGTGTACAATAAATGT-3′, SEQ ID NO: 110; and complementary strand: 5′-GTTCCAGCGGATCCGGATAC-3′, SEQ ID NO:111.

The library fragments were synthesized and amplified by PCR (PCR superMix HiFi, Invitrogen) with pM197 as template. This PCR product was then double-digested with SphI/NotI and cloned into SphI/NotI-digested pM197 phagemid vector. The resulting ligation reaction was electroporated into electrocompetent Escherichia coli TG1 cells (Lucigen, Wis.) according to Engberg et al. and manufacture suggestion), yielding a library size of 6.2×10⁸. The library was stored as glycerol stocks, rescued and used for phage production according to standard protocols.

### Library Characterization

A total of 96 clones were randomly picked and sequenced to verify the insert size, frame shift and diversity of each position of the 7 amino acid region. The display of the CD59-pIII fusion protein on the phage surface was evaluated by Western blot. Anti-pIII mouse mAb (NEB, 1:1000 dilution) and HRP conjugated goat anti-mouse IgG (Jackson Lab, 1:6000) were used as detecting reagents.

### Library Panning on Microtiter Plates

Panning against GPIIb/IIIa was performed. Immulon 1B plate was coated with 100 μl/well of GPIIb/IIIa protein (5 μg/ml in coating buffer, see GPIIb/IIIa binding assay method) overnight at 4° C. followed by two short rinses with BB buffer and blocked with blocking buffer (BB buffer with 3% BSA) for 2 hours at room temperature (RT). After rinsing with BB buffer, 10″ phage particles in blocking buffer were added per well and incubated RT for 2 hours. Unbounded phage was washed away by 5 times with BBT (0.1% Tween 20) and 5 times with BB. The bound phage was eluted with 100 μl 0.1M glycine elution buffer per well and then neutralized with 10 μl of 1 M Tris. The eluted phage was then used to infect exponentially growing TG1 cells.

For Hyperphage derived library, three rounds of microtiter plate panning and one round of solution panning were carried. Phage ELISA against GPIIb/IIIa was performed to screen for positive clones.

### Library Panning in Solution

GPIIb/IIIa was biotinylated using EZ-link NHS-Biotin (Pierce, Rockford Ill.) according to the manufacturer's instruction. The biotinylated GPIIb/IIIa (1^{St} round panning 50 nM; 2^{nd} round 20 nM and 3^{rd} round 5 nM) was incubated with pre-blocked 7×10″ cfu (1^{st} round panning, 1×10¹¹ cfu phage for 2^{nd} and 3rd) phage CD59/F2/7mer library for 1 hour on a rotator at room temperature. The phage-target mixture was captured on pre-blocked strepavidin-coupled magnetic beads by incubating another 10 minutes at room temperature. Separation of beads with solutions was performed with a Magnetic Concentrator. The beads then were washed with BBT 0.1% for 4 times, followed by 5 times BB for the first round panning The washing stringency was increased for the 2^{nd} and 3^{rd} roundpanning Bound phage was eluted by incubation with 0.1M Glycine buffer pH2.5 for 15 minutes and then neutralized with 1M Tris. The eluted phage was then used to infect exponentially growing TG1 cells.

For the M13K07 derived library, three rounds of solution panning were performed.

### ELISA Screening

After three to four rounds of panning, the eluted phage was used to infect exponentially growing HB2151 cells or DH10BF′. Individual clones were inoculated in 200 ul of 2xYT with 100 μg/ml ampicillin and 0.1% glucose in 96-well plates. The plates were incubated for 3 hours at 37° C. in a shaker incubator. The cells were then induced with 1 mM IPTG for 3-4 hours at 30° C. Periplasmic fraction was extracted and tested in GPIIb/IIIa ELISA.

### Example 3

### Expression and Testing of CD59 Wt and F2 RGD Variants

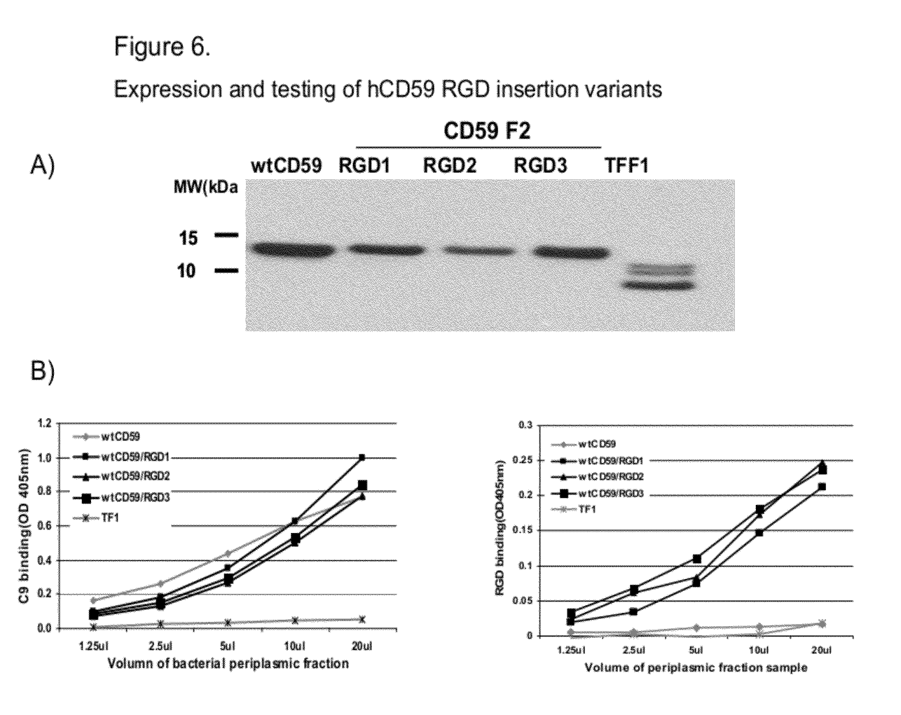

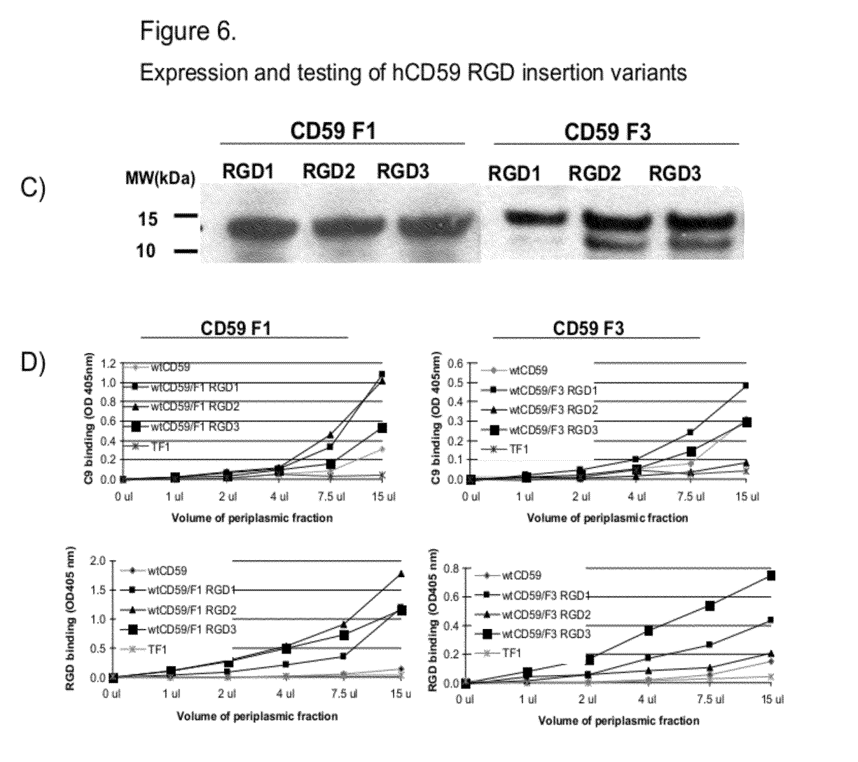

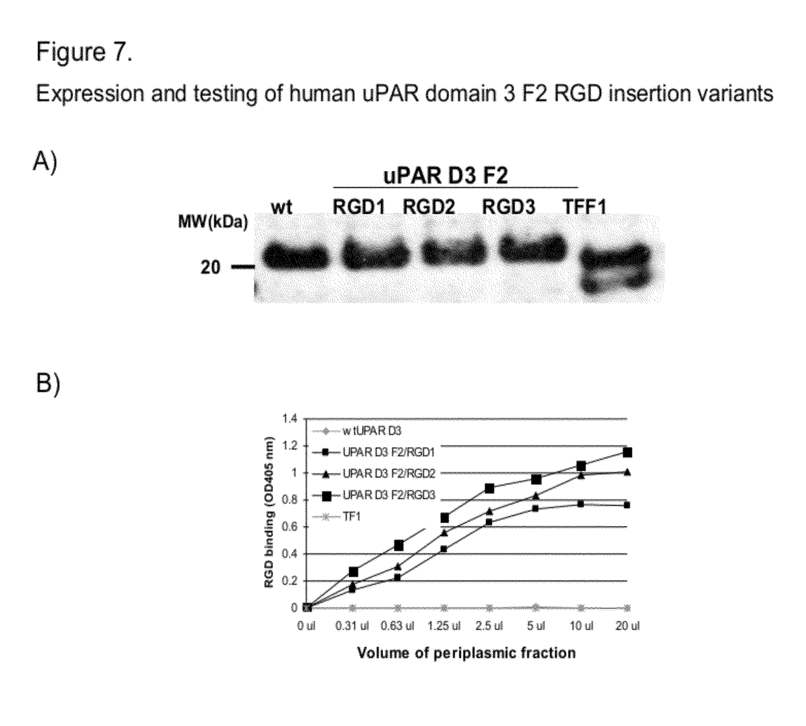

In order to test the ability of the human TFPD to serve as a scaffold for incorporating novel binding activity a series of peptides derived from the disintegrin, eristostain, containing the integrin recognition sequence, Arginine-Glycine-Aspartic acid (RGD), were engineered into each of the fingers of hCD59 (FIG. 5). The RGD containing peptides were of variable length (7, 9, or 11 amino acids) and were incorporated into the tips of the fingers as determined from the hCD59 structure. The hCD59 RGD containing loop insertion variants were expressed in E. coli as soluble FLAG-tagged proteins, and tested for CD59 activity (binding to complement factor C9) and for integrin binding in a GPIIb/IIIa ELISA (FIG. 6). FIG. 6A is an SDS-PAGE gel immunoblot of extracts from the periplasmic fraction of E. coli, expressing hCD59 wild type (wt) and three hCD59 RGD variants (RGD 1, 2, and 3) inserted within finger 2 (F2). The wild type and variants are secreted into the periplasm as single species of approximately 13 kDa. FIG. 6B shows the testing of the hCD59 wt and F2 RGD variants in the C9 and GPIIb/IIIa binding ELISAs. Both the CD59 wt and the RGD containing variants bind C9 in a dose dependent manner indicating that all of these CD59 species have retained proper folding and display native CD59 activity. In the GPIIb/IIIa ELISA the RGD variants bind the integrin in a dose dependent manner, while the CD59 wt displays negligible binding, as expected. This supports the notion that the CD59 scaffold can be engineered to display a novel binding activity within the F2 loop, while retaining native C9 binding activity. Similar results have been obtained by engineering the same RGD loop insertion sequences into the F1 and F3 loop regions of hCD59 (FIGS. 6C and 6D). In addition the same RGD loop sequences have been cloned into the F2 loop of the uPAR domain 3 (uPARD3). The E. coli expressed proteins were tested for GPIIb/IIIa binding and exhibited similar binding (FIG. 7).

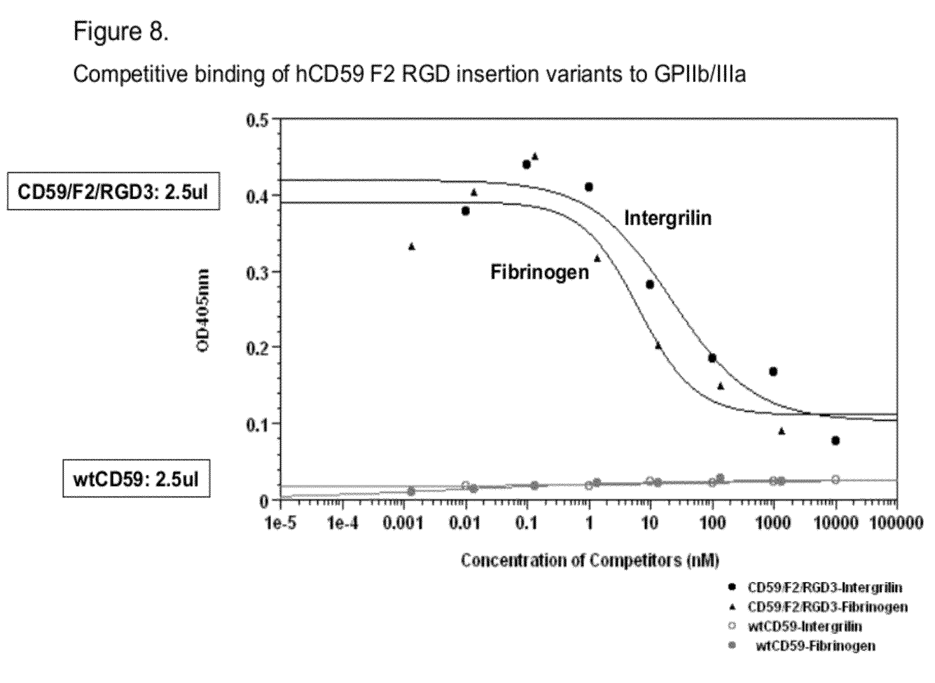

In order to show the specificity of the CD59 RGD variants for integrin binding a competitive ELISA was performed in which a fixed amount of hCD59 wt or the CD59 F2 RGD3 variant were bound to GPIIb/IIIa and competed with the natural ligand, fibrinogen, or the antagonist, Integrilin® (FIG. 8). In each case there was a dose dependent competition for IIb/IIIa binding, indicating that the insertion variant binds the integrin with high specificty for the RGD binding site.

The RGD loop insertion studies demonstrate that the CD59 scaffold is capable of accommodating modification within the F1, F2, and F3 loops, and supports the concept for using the human TFPD as a mimetics scaffold. The data demonstrate the flexibility of a CD59 or uPAR domain 3 scaffold and the ability to engineer novel binding functionality into the F2 loop.

### Example 4

### Human CD59 wt and RGD Variants can be Displayed on Phage

The next step in validating TFPDs as a mimetics scaffold was to demonstrate the ability to present the scaffold by a display method for screening libraries, for example, ribosome, bacterial, yeast, or mammalian display. Among these methods bacterial display using phage is the most popular and straightforward technique for constructing and screening libraries.

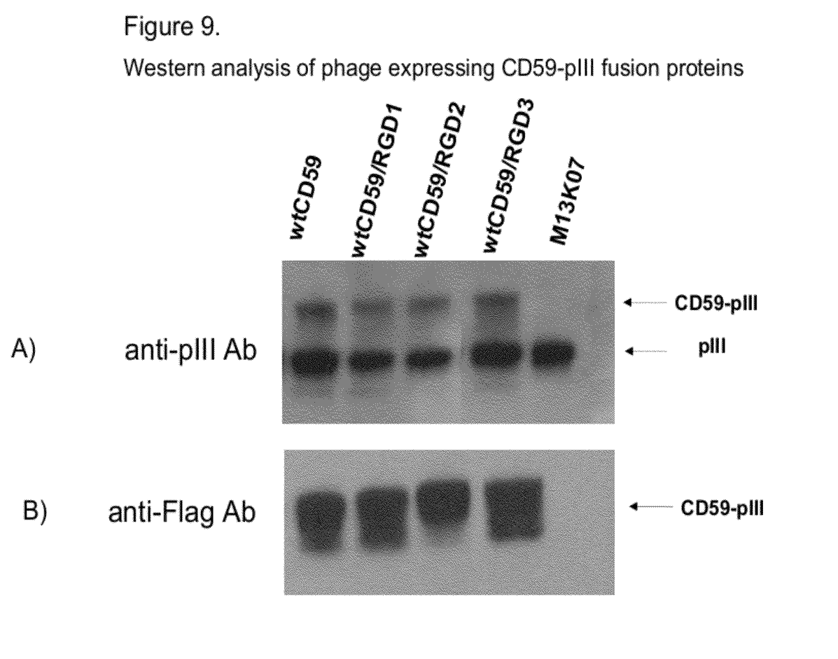

The FLAG-tagged hCD59 wt and F2 RGD variants were cloned into a phagemid vector and expressed in phage infected E. coli as fusion proteins with the phage gIII protein (CD59-gIII). CD59-gIII expressing phage were isolated from infected E. coli following an overnight culture and analyzed by SDS-PAGE (FIG. 9). The CD59-gIII fusion protein was detected with an anti-gIII antibody traveling at a slightly higher molecular weight than the gIII protein (FIG. 9A). The lower intensity of the fusion protein band relative to the gIII band is expected since the CD59-gIII protein is expressed at a lower level than the gIII protein expressed by the helper phage. The anti-FLAG antibody detects a strong band for the CD59 wt and the three RGD variant containing fusion proteins (FIG. 9B).

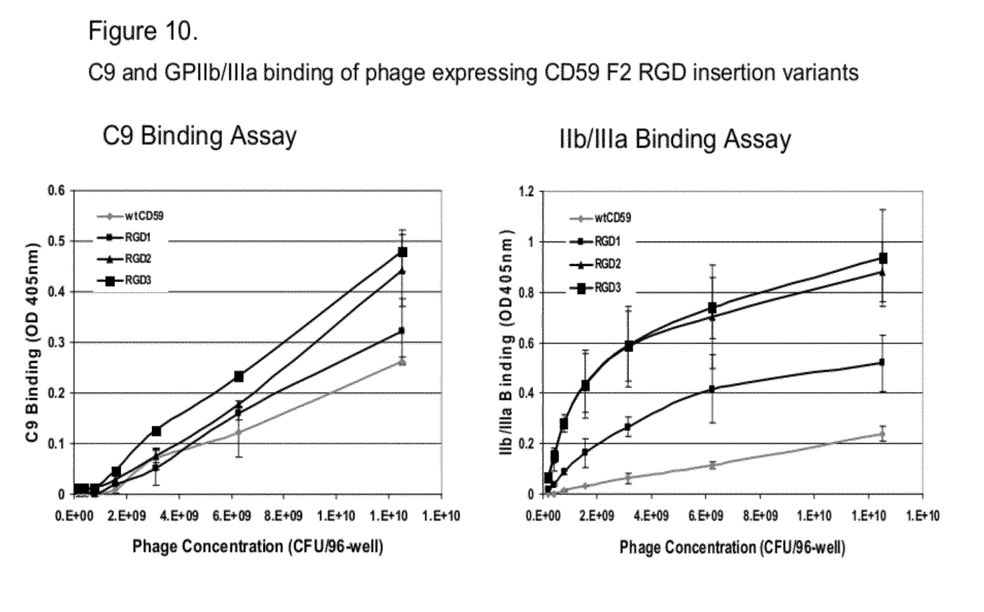

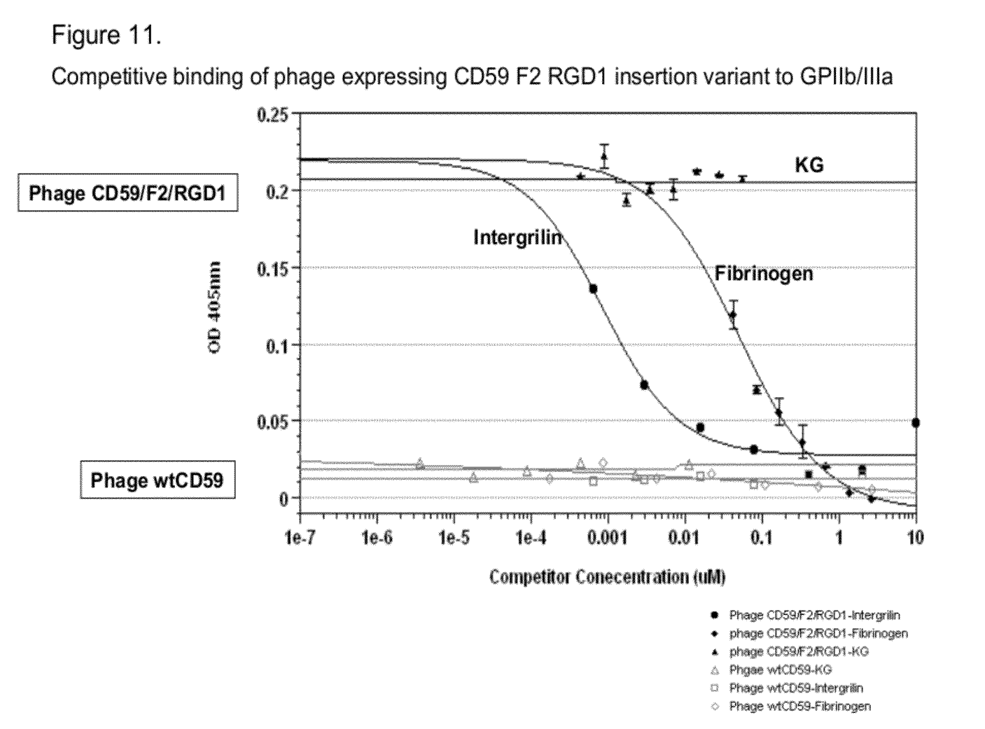

The CD59-gIII expressing phage were shown to bind both C9 as well as GPIIB/IIIa in the binding ELISAs, indicating that the proteins were folded properly and capable of displaying both wild type and RGD binding activities (FIG. 10). Similar to the results with the soluble CD59 wt and F2 RGD variant proteins the binding of the CD59-gIII expressing phage is dose dependent, and in the case of the phage expressing the CD59-gIII RGD variants, the binding to GPIIb/IIIa is specific for the RGD binding site, as evidenced by the competitive displacement from the integrin with known GPIIb/IIA ligands (FIG. 11).

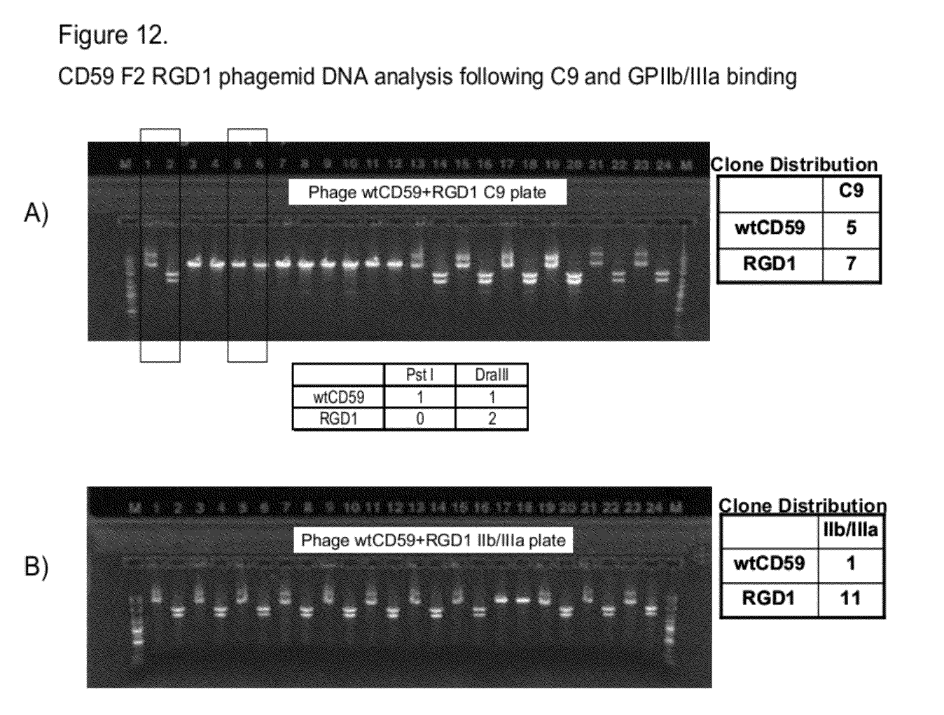

In order to assess the ability to select phage expressing specific binders, equal amounts of CD59 wt and CD59 F2 RGD 1 expressing phage were mixed and then bound to either C9 or GPIIb/IIIa using the same ELISA assay format. The plates were washed, the bound phage were eluted and used to infect TG1 E. coli. Phagemid DNA analysis of 12 clones from each plate showed that an approximately equal proportion of CD59 wt and CD59 F2 RGD1 expressing phage bound to the C9 plate (5 wt and 7 RGD clones) (FIG. 12A). However, the phage binding to the GPIIb/IIIa plate contained almost exclusively RGD expressing phage (1 wt and 11 RGD clones, FIG. 12B). The results demonstrate that the CD59 wt and RGD variant proteins retain their binding properties when displayed on phage as gIII fusion proteins.

### Example 5

### Construction and Validation of a CD59 F2 Library

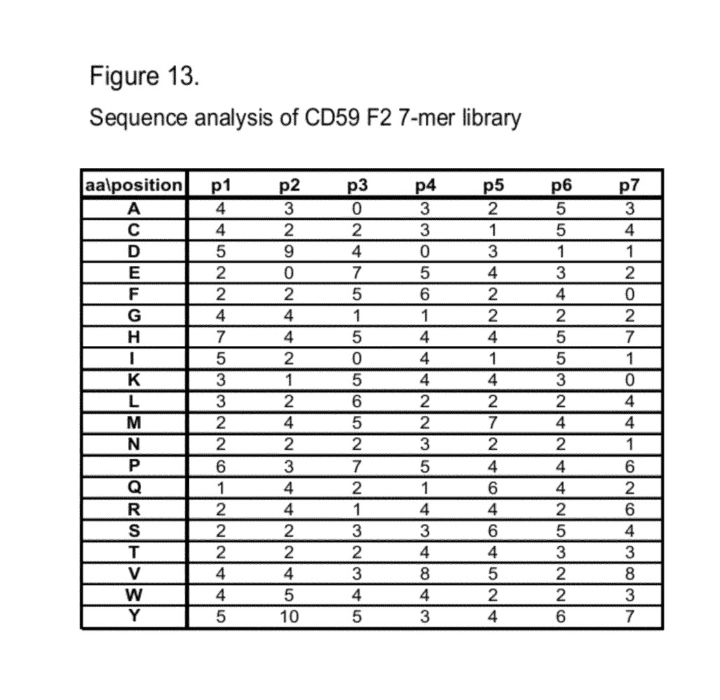

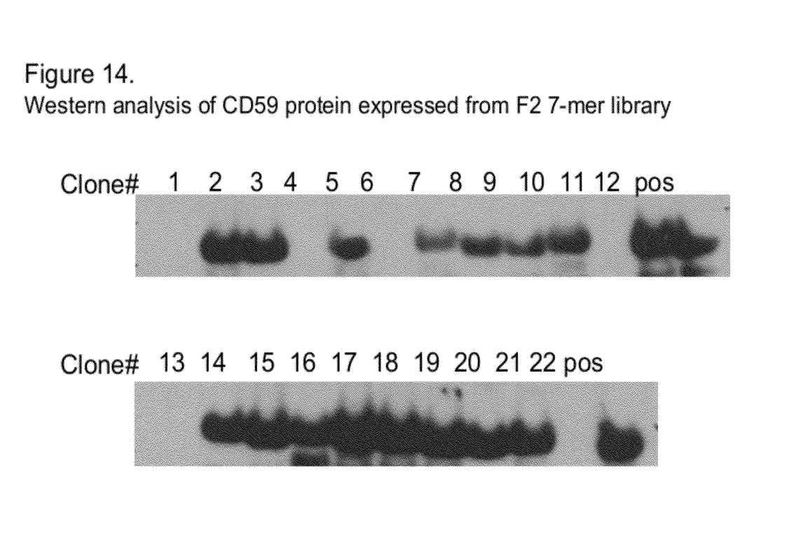

A TFPD library was designed and constructed within the tip of the F2 loop of hCD59 by replacing Gly32-Ala33 with an insert of seven amino acids of random composition. The design was similar to the CD59 F2 RGD1 variant in which a seven amino acid peptide was inserted within the F2 loop, except in this case the composition of the 7-mer was randomized with all possible 20 amino acids occurring at each position. Triphosphoramidite chemistry was used for synthesis of the random oligonucleotides to minimize frame shifts, eliminate the appearance of stop codons, and provide equal representation of all twenty amino acids, including cysteine. The theoretical diversity of the F2 7-mer library is (20)⁷ or approximately 1.28×10⁹. The random oligonucleotide mix was amplified by PCR and cloned into the pM197 phagemeid vector. Random clones were isolated from TG1 E. coli transformed with vector DNA and characterized. The size of the library was determined to be 6.2×10⁸ by limiting dilution following transformation of TG1 cells. Sequence analysis of 69 clones showed a predicted random distribution of amino acids at each position (FIG. 13). The number of clones from the library expressing soluble CD59 was determined to be approximately 72% by western analysis of periplasmic extracts following transformation HB2151 cells (16 out of 22 clones expressing protein) (FIG. 14).

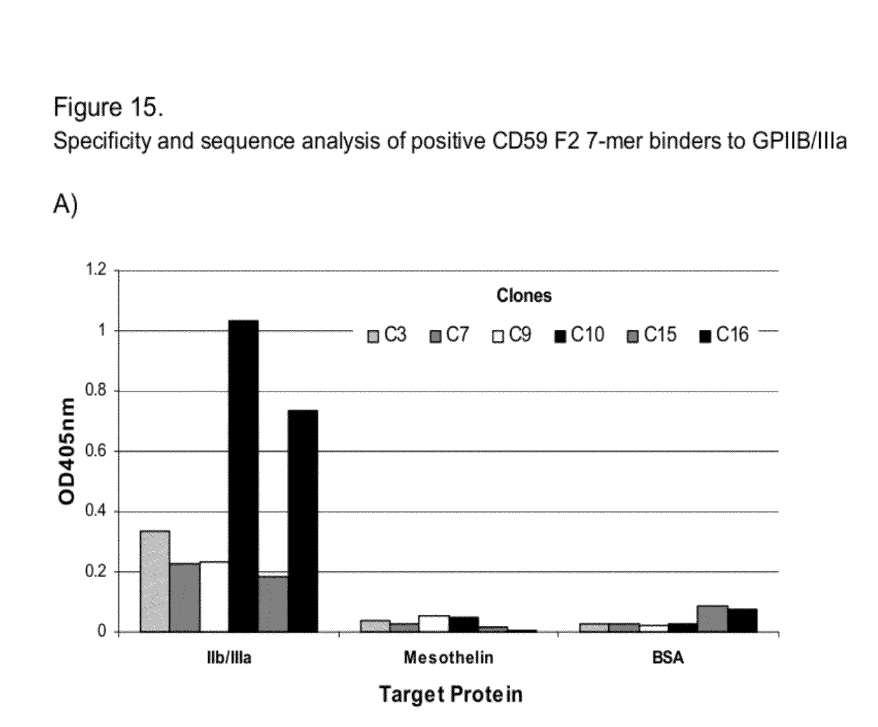

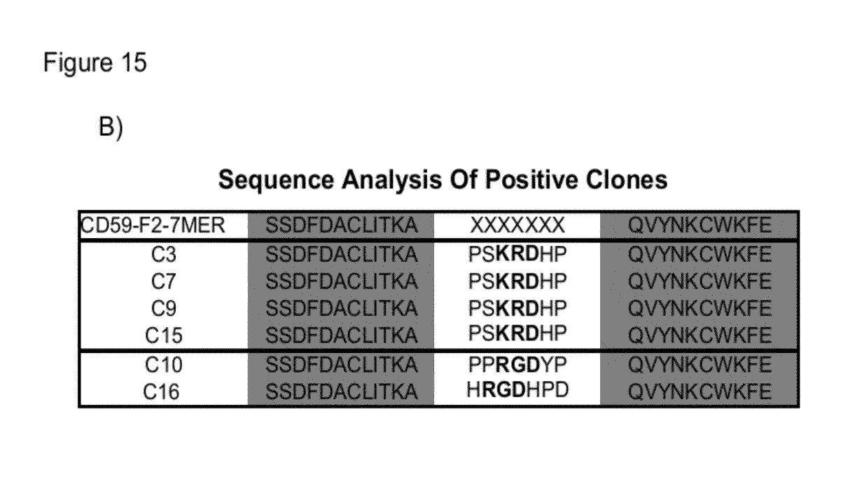

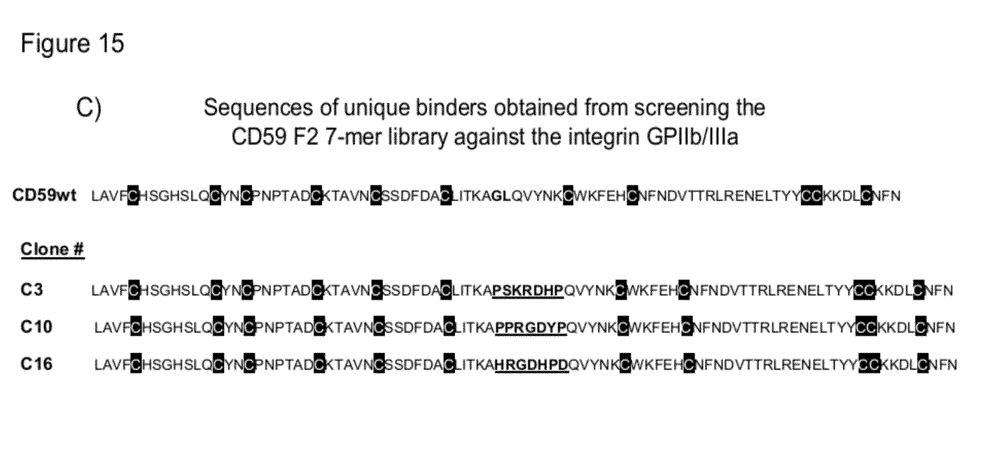

The CD59 F2 7-mer library was screened against GPIIb/IIIa and following three rounds of panning a selected number of the binders were verified for specificity (FIG. 15A). Six of the clones were sequenced and revealed the known integrin binding RGD-motif (FIG. 15B). The full length sequences, SEQ ID NO:112-114 respectively, for the unique binders obtained from screening the CD59 F2 7-mer library against IIb/IIIa are shown in FIG. 15C. This data demonstrates the capability of the CD59 F2 library to screen and identify potent binders of selected targets, and validates the human TFPD as an antibody mimetics scaffold.

### Example 6

### Construction and Validation of a CD59 F1 library

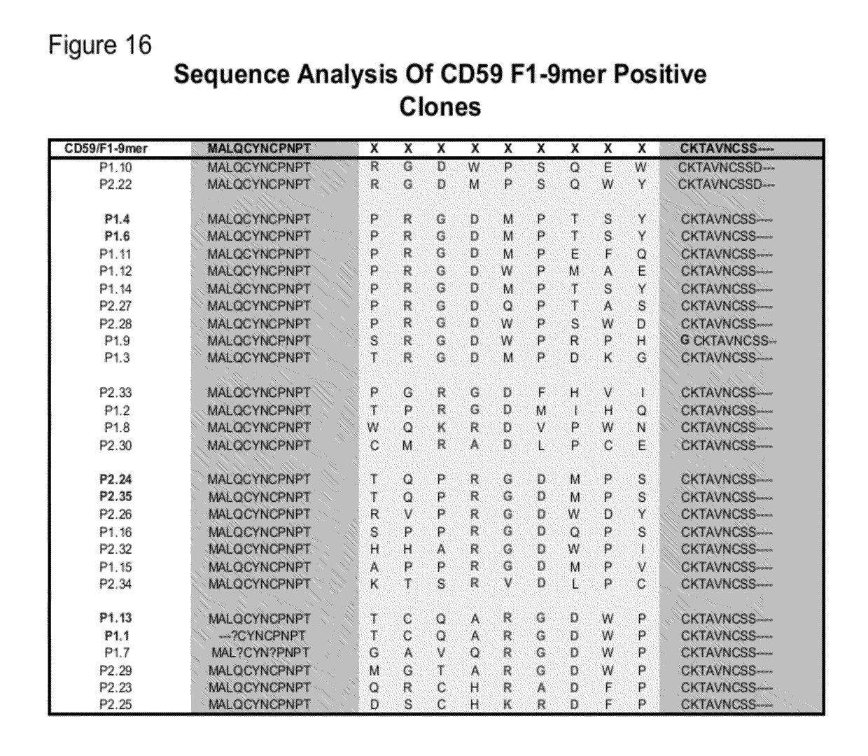

A TFPD library was designed and constructed within the tip of the F1 loop of hCD59 by replacing Ala12-Asp 13 with an insert of nine amino acids of random composition. The design was similar to the CD59 F1 RGD2 variant in which a nine amino acid RGD containing sequence derived from eristostatin was inserted within the F1 loop, except in this case the composition of the 9-mer was randomized with all possible 20 amino acids occurring at each position. The theoretical diversity of the F19-mer library is (20)⁹ or approximately 5.12×10″. The CD59 F19-mer library was screened against GPIIb/IIIa and following three rounds of panning a selected number of the binders were verified for specificity. Positive clones were sequenced and revealed twenty-five unique sequences containing the known integrin binding RGD-motif (FIG. 16). This data demonstrates the capability of generating highly diverse libraries using the F1 loop within the hCD59 TFPD and the ability to screen and identify potent binders of selected targets with the F1 library.

### Example 7

### Construction and Validation of a UPARD3 F2 Library

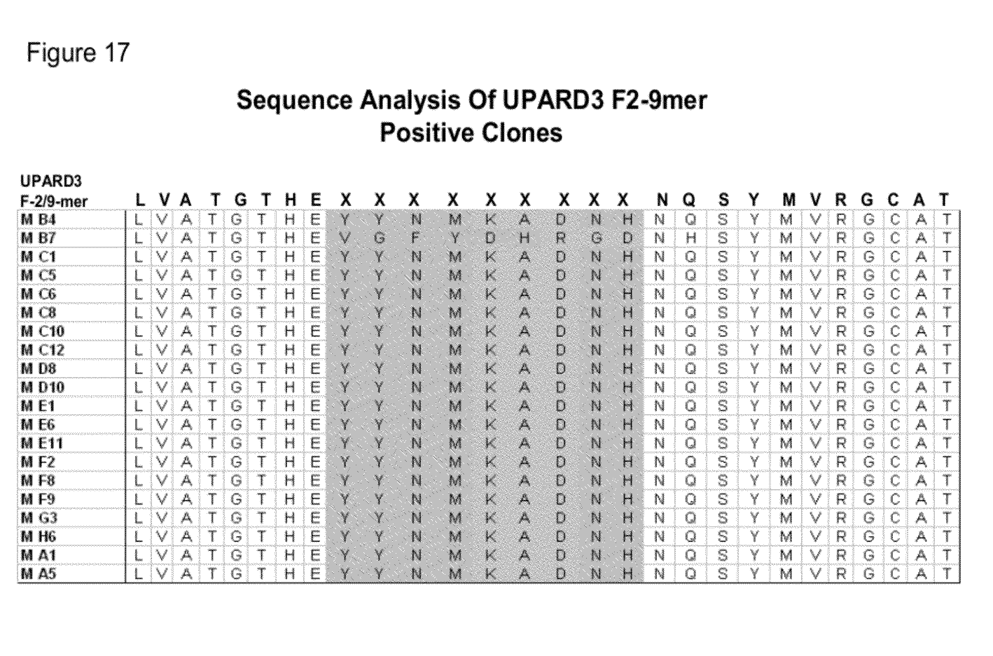

A TFPD library was designed and constructed within the tip of the F2 loop of human UPARD3 by replacing Pro40-Lys41 within the third domain of human UPAR (where residue 1 of UPARD3 corresponds to residue 192 of the full length UPAR) with an insert of nine amino acids of random composition. The design was similar to the UPARD3 F2 RGD2 variant in which a nine amino acid RGD containing sequence was inserted within the F2 loop, except in this case the composition of the 9-mer was randomized with all possible 20 amino acids occurring at each position. The theoretical diversity of the F2 9-mer library is (20)⁹ or approximately 5.12×10″. The UPARD3 F2 9-mer library was screened against GPIIb/IIIa and following three rounds of panning a selected number of the binders were verified for specificity. The positive clones were sequenced and revealed two unique sequences (see clone sequences M B4 and M B7) containing the known integrin binding RGD-motif (FIG. 17). This data demonstrates the capability of the hUPARD3 F2 library to screen and identify potent binders of selected targets, and validates the utility of the TFPD as a mimetics scaffold using different members of this protein family.

All publications and patents mentioned in the above specification are incorporated herein by reference. Various modifications and variations of the described methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the above-described modes for carrying out the invention which are obvious to those skilled in the art are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A polypeptide comprising a three finger protein domain (TFPD) wherein said TFPD has an amino acid sequence that has been modified relative to the amino acid sequence of a naturally occurring TFPD such that the modified TFPD binds to a specified target molecule.

2. The polypeptide of claim 1, wherein said modification is at a position in finger 1 (F1) of said TFPD.

3. The polypeptide of claim 1, wherein said modification is at a position in finger 2 (F2) of said TFPD.

4. The polypeptide of claim 1, wherein said modification is at a position in finger 3 (F3) of said TFPD.

5. The polypeptide of claim 1, wherein said modifications are made in a combination of two or more fingers selected from the group consisting of F1, F2, and F3.

6. The polypeptide of any of claims 1-5, wherein said amino acid sequence is modified by one or more substitutions.

7. The polypeptide of claim 6, wherein said substitution comprises a random amino acid residue.

8. The polypeptide of any of claims 1-5, wherein said amino acid sequence is modified by an insertion.

9. The polypeptide of claim 8, wherein said insertion is a random amino acid sequence.

10. The polypeptide of claim 8, wherein said insertion is a predetermined sequence.

11. The polypeptide of any of the preceding claims, wherein the TFPD is selected from the group consisting of CD59, urokinase receptor (uPAR) domain 1, uPAR domain 2, uPAR domain 3, TGFR domain 1, TGFR domain 2, ACVR1, ACV1B, ACV1C, ACVL1, AMHR2, AVR2A, AVR2B, EMR1B, EMR1A, EMPR2, LYPD1, LYPD2, LYPD3-1, LYPD3-2, LYPD4-1, LYPD4-2, LYPD5-1, LYPD5-2 LYPD6, LPD6B, LY6E, LY6D, LY6DL, LY66C, LY6K, LYG6E, LY65C, LY65B, LY66D, LY6H, LYNX1, PATE, PATEB, PATEDJ, PATEM, PSCA, SLUR1, SLUR2, ASPX, HDBP1, SACA4, C9orf57, TX101-1, TX101-2, CD177-1, CD177-2, CD177-3, CD177-4, and BAMBI.

12. The polypeptide of claim 11, wherein the TFPD is CD59.

13. The polypeptide of claim 12, wherein the CD59 is from a species selected from the group consisting of human, owl monkey, marmoset, African green monkey, crab-eating macaque, baboon, orangutan, squirrel monkey, chimpanzee, rabbit, pig, rat and mouse.

14. The polypeptide of claim 12, wherein the CD59 is an inactive mutant.

15. The polypeptide of claim 14, wherein the CD59 inactive mutant comprises a modification at position 24 or 40.

16. The polypeptide of claim 11, wherein the TFPD is uPAR domain 3.

17. The polypeptide of claim 16, wherein the uPAR domain 3 is from a species selected from the group consisting of human, owl monkey, marmoset, African green monkey, crab-eating macaque, baboon, orangutan, squirrel monkey, chimpanzee, rabbit, pig, rat and mouse.

18. The polypeptide of claim 16, wherein the uPAR domain 3 is an inactive mutant.

19. The polypeptide of claim 18, wherein the uPAR domain 3 inactive mutant comprises a modification at position 245.

20. The polypeptide of claim 1, wherein the specified target is not bound by the naturally occurring TFPD.

21. The polypeptide of claim 1, wherein the specified target is selected from the group consisting of a protein, a nucleotide, an antibody, a small molecule and an antigen of interest.

22. The polypeptide of claim 1, further comprising an element imparting effector function.

23. The polypeptide of claim 22, wherein the element extends circulation half-life.

24. The polypeptide of claim 22, wherein the element allows chemical conjugation.

25. An isolated polynucleotide encoding the polypeptide of any of claims 1-21.

26. An expression vector comprising a nucleotide sequence as defined in claim 25.

27. A host cell comprising an expression vector as defined in claim 26.

28. A pharmaceutical composition comprising a polypeptide as defined in claim 1 and at least one pharmaceutically acceptable carrier or excipient.

29. A library of polypeptides comprising a plurality of three finger protein domains (TFPD) wherein said TFPD have amino acid sequences that has been modified relative to the amino acid sequences of corresponding naturally occurring TFPD such that the modified TFPD binds to a specified target molecule.

30. The library of claim 29, wherein said amino acid sequences are randomly modified.

31. The library of claim 29, wherein the library comprises at least 100 different polypeptides comprising different modified TFPDs.

32. A library of polynucleotides encoding the library of polypeptides of claim 29.

33. A multi-specific polypeptide comprising a three finger protein domain (TFPD) wherein said TFPD has an amino acid sequence that has been modified relative to the amino acid sequence of a naturally occurring TFPD such that the modified TFPD binds to two or more specified target molecules.

34. The multi-specific polypeptide of claim 33, wherein the target molecules bind to different fingers of the modified TFPD.

35. A multi-specific compound comprising a fusion of two or more three finger protein domains (TFPDs) wherein said TFPDs have amino acid sequences that have been modified relative to the amino acid sequence of naturally occurring TFPDs such that the modified TFPDs binds different target molecules.

36. A method of using a three fingered protein domain (TFPD) as a scaffold for generating antibody mimetics comprising inserting an amino acid sequence into one or more fingers of the TFPD.

37. The method of claim 36, wherein the TFPD is CD59.

38. The method of claim 36, wherein the TFPD is uPAR domain 3.

39. The method of claim 36, wherein the amino acid sequence is a random sequence.

40. A polypeptide comprising a three finger protein domain (TFPD) wherein said TFPD has an amino acid sequence that has been modified relative to the amino acid sequence of a naturally occurring TFPD such that the modified TFPD binds to GPIIb/IIIa.

41. The polypeptide of claim 40, wherein said TFPD has an amino acid sequence as shown in SEQ ID NO:112.

42. The polypeptide of claim 40, wherein said TFPD has an amino acid sequence as shown in SEQ ID NO:113.

43. The polypeptide of claim 40, wherein said TFPD has an amino acid sequence as shown in SEQ ID NO:114.


SEQUENCE LISTING (114 sequences)

SEQ ID NO: 1 - protein, 70 aa, Homo sapiens
LQCYNCPNPTADCKTAVNCSSDFDACLITKAGLQVYNKCWKFEHCNFNDVTTRLRENELTYYCCKKDLCN

SEQ ID NO: 2 - protein, 77 aa, Homo sapiens
LRCMQCKTNGDCRVEECALGQDLCRTTIVRLWEEGEELELVEKSCTHSEKTNRTLSYRTGLKITSLTEVVCGLDLCN

SEQ ID NO: 3 - protein, 85 aa, Homo sapiens
LECISCGSSDMSCERGRHQSLQCRSPEEQCLDVVTHWIQEGEEGRPKDDRHLRGCGYLPGCPGSNGFHNNDTFHFLKCCNTTKCN

SEQ ID NO: 4 - protein, 81 aa, Homo sapiens
RQCYSCKGNSTHGCSSEETFLIDCRGPMNQCLVATGTHEPKNQSYMVRGCATASMCQHAHLGDAFSMNHIDVSCCTKSGCN

SEQ ID NO: 5 - protein, 82 aa, Homo sapiens
CYSCVQKADDGCSPNKMKTVKCAPGVDVCTEAVGAVETIHGQFSLAVRGCGSGLPGKNDRGLDLHGLLAFIQLQQCAQDRCN

SEQ ID NO: 6 - protein, 83 aa, Homo sapiens
CYSCVGLSREACQGTSPPVVSCYNASDHVYKGCFDGNVTLTAANVTVSLPVRGCVQDEFCTRDGVTGPGFTLSGSCCQGSRCN

SEQ ID NO: 7 - protein, 104 aa, Homo sapiens
CLLGAISTLPRAGALLCYEATASRFRAVAFHNWKWLLMRNMVCKLQEGCEETLVFIETGTARGVVGFKGCSSSSSYPAQISYLVSPPGVSIASYSRVCRSYLCN

SEQ ID NO: 8 - protein, 82 aa, Homo sapiens
CPTCVGEHMKDCLPNFVTTNSCPLAASTCYSSTLKFQAGFLNTTFLLMGCAREHNQLLADFHHIGSIKVTEVLNILEKSQIV

SEQ ID NO: 9 - protein, 97 aa, Homo sapiens
LFGAALCLTGSQALQCYSFEHTYFGPFDLRAMKLPSISCPHECFEAILSLDTGYRAPVTLVRKGCWTGPPAGQTQSNADALPPDYSVVRGCTTDKCN

SEQ ID NO: 10 - protein, 80 aa, Homo sapiens
CYACIGVHQDDCAIGRSRRVQCHQDQTACFQGNGRMTVGNFSVPVYIRTCHRPSCTTEGTTSPWTAIDLQGSCCEGYLCN

SEQ ID NO: 11 - protein, 88 aa, Homo sapiens
CQKGLSMTVEADPANMFNWTTEEVETCDKGALCQETILIIKAGTETAILATKGCIPEGEEAITIVQHSSPPGLIVTSYSNYCEDSFCN

SEQ ID NO: 12 - protein, 76 aa, Homo sapiens
CPTCVALGTCFSAPSLPCPNGTTRCYQGKLEITGGGIESSVEVKGCTAMIGCRLMSGILAVGPMFVREACPHQLLT

SEQ ID NO: 13 - protein, 87 aa, Homo sapiens
QFGTVQHVWKVSDLPRQWTPKNTSCDSGLGCQDTLMLIESGPQVSLVLSKGCTEAKDQEPRVTEHRMGPGLSLISYTFVCRQEDFCN

SEQ ID NO: 14 - protein, 77 aa, Homo sapiens
CPVCLSMEGCLEGTTEEICPKGTTHCYDGLLRLRGGGIFSNLRVQGCMPQPGCNLLNGTQEIGPVGMTENCNRKDFL

SEQ ID NO: 15 - protein, 89 aa, Homo sapiens
HRGTTIMTHGNLAQEPTDWTTSNTEMCEVGQVCQETLLLLDVGLTSTLVGTKGCSTVGAQNSQKTTIHSAPPGVLVASYTHFCSSDLCN

SEQ ID NO: 16 - protein, 53 aa, Homo sapiens
CPTCVQPLGTCSSGSPRMTCPRGATHCYDGYIHLSGGGLSTKMSIQGCVAQPS

SEQ ID NO: 17 - protein, 74 aa, Homo sapiens
LQCFCHLCTKDNFTCVTDGLCFVSVTETTDKVIHNSMCIAEIDLIPRDRPFVCAPSSKTGSVTTTYCCNQDHCN

SEQ ID NO: 18 - protein, 96 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSDECN

SEQ ID NO: 19 - protein, 68 aa, Homo sapiens
YMCVCEGLSCGNEDHCEGQQCFSSLSINDGFHVYQKGCFQVYEQGKMTCKTPPSPGQAVECCQGDWCN

SEQ ID NO: 20 - protein, 71 aa, Homo sapiens
LLCACTSCLQANYTCETDGACMVSIFNLDGMEHHVRTCIPKVELVPAGKPFYCLSSEDLRNTHCCYTDYCN

SEQ ID NO: 21 - protein, 68 aa, Homo sapiens
LKCVCLLCDSSNFTCQTEGACWASVMLTNGKEQVIKSCVSLPELNAQVFCHSSNNVTKTECCFTDFCN

SEQ ID NO: 22 - protein, 66 aa, Homo sapiens
LVTCTCESPHCKGTCRGAWCTVVLVREEGRHPQEHRGCGNLHRELCRGRPTEFVNHYCCDSHLCNH

SEQ ID NO: 23 - protein, 65 aa, Homo sapiens
IRCLYSRCCFGIWNLTQDRAQVEMQGCRDSDEPGCESLHCDPSPRAHPSPGSTLFTCSCGTDFCN

SEQ ID NO: 24 - protein, 98 aa, Homo sapiens
ISCSSGAILGRSETQECLFFNANWEKDRTNQTGVEPCYGDKDKRRHCFATWKNISGSIEIVKQGCWLDDINCYDRTDCVEKKDSPEVYFCCCEGNMCN

SEQ ID NO: 25 - protein, 97 aa, Homo sapiens
SLCAGSGRGEAETRECIYYNANWELERTNQSGLERCEGEQDKRLHCYASWRNSSGTIELVKKGCWLDDFNCYDRQECVATEENPQVYFCCCEGNFCN

SEQ ID NO: 26 - protein, 73 aa, Homo sapiens
LKCYCSGHCPDDAINNTCITNGHCFAIIEEDDQGETTLASGCMKYEGSDFQCKDSPKAQLRRTIECCRTNLCN

SEQ ID NO: 27 - protein, 74 aa, Homo sapiens
LRCKCHHHCPEDSVNNICSTDGYCFTMIEEDDSGLPVVTSGCLGLEGSDFQCRDTPIPHQRRSIECCTERNECN

SEQ ID NO: 28 - protein, 93 aa, Homo sapiens
RLCAFKDPYQQDLGIGESRISHENGTILCSKGSTCYGLWEKSKGDINLVKQGCWSHIGDPQECHYEECVVTTTPPSIQNGTYRFCCCSTDLCN

SEQ ID NO: 29 - protein, 83 aa, Homo sapiens
CYQCEEFQLNNDCSSPEFIVNCTVNVQDMCQKEVMEQSAGIMYRKSCASSAACLIASAGYQSFCSPGKLNSVCISCCNTPLCN

SEQ ID NO: 30 - protein, 76 aa, Homo sapiens
CYVCPEPTGVSDCVTIATCTTNETMCKTTLYSREIVYPFQGDSTVTKSCASKCKPSDVDGIGQTLPVSCCNTELCN

SEQ ID NO: 31 - protein, 82 aa, Homo sapiens
FKCFTCEKAADNYECNRWAPDIYCPRETRYCYTQHTMEVTGNSISVTKRCVPLEECLSTGCRDSEHEGHKVCTSCCEGNICN

SEQ ID NO: 32 - protein, 82 aa, Homo sapiens
FKCFTCENAGDNYNCNRWAEDKWCPQNTQYCLTVHHFTSHGRSTSITKKCASRSECHFVGCHHSRDSEHTECRSCCEGMICN

SEQ ID NO: 33 - protein, 79 aa, Homo sapiens
LMCFSCLNQKSNLYCLKPTICSDQDNYCVTVSASAGIGNLVTFGHSLSKTCSPACPIPEGVNVGVASMGISCCQSFLCN

SEQ ID NO: 34 - protein, 73 aa, Homo sapiens
LRCHVCTSSSNCKHSVVCPASSRFCKTTNTVEPLRGNLVKKDCAESCTPSYTLQGQVSSGTSSTQCCQEDLCN

SEQ ID NO: 35 - protein, 83 aa, Homo sapiens
LRCHDCAVINDFNCPNIRVCPYHIRRCMTISIRINSRELLVYKNCTNNCTFVYAAEQPPEAPGKIFKTNSFYWVCCCNSMVCN

SEQ ID NO: 36 - protein, 79 aa, Homo sapiens
IRCHSCYKVPVLGCVDRQSCRLEPGQQCLTTHAYLGKMWVFSNLRCGTPEEPCQEAFNQTNRKLGLTYNTTCCNKDNCN

SEQ ID NO: 37 - protein, 82 aa, Homo sapiens
VWCHVCERENTFECQNPRRCKWTEPYCVIAAVKIFPRFFMVAKQCSAGCAAMERPKPEEKRFLLEEPMPFFYLKCCKIRYCN

SEQ ID NO: 38 - protein, 68 aa, Homo sapiens
LRCYICGFTKPCHPVPTECRDDEACGISIGTSGKSCLSRAQCPLPGYATYWLHSYTLWHHCCEQDLCN

SEQ ID NO: 39 - protein, 81 aa, Homo sapiens
LRCYRCLLETKELGCLLGSDICLTPAGSSCITLHKKNSSGSDVMVSDCRSKEQMSDCSNTRTSPVSGFWIFSQYCFLDFCN

SEQ ID NO: 40 - protein, 80 aa, Homo sapiens
RTCHFCLVEDPSVGCISGSEKCTISSSSLCMVITIYYDVKVRFIVRGCGQYISYRCQEKRNTYFAEYWYQAQCCQYDYCN

SEQ ID NO: 41 - protein, 82 aa, Homo sapiens
MRCYNCGGSPSSSCKEAVTTCGEGRPQPGLEQIKLPGNPPVTLIHQHPACVAAHHCNQVETESVGDVTYPAHRDCYLGDLCN

SEQ ID NO: 42 - protein, 66 aa, Homo sapiens
LWCQDCTLTTNSSHCTPKQCQPSDTVCASVRITDPSSSRKDHSVNKMCASSCDFVKRHFFSDYLMG

SEQ ID NO: 43 - protein, 72 aa, Homo sapiens
LDCHVCAYNGDNCFNPMRCPAMVAYCMTTRTYYTPTRMKVSKSCVPRCFETVYDGYSKHASTTSCCQYDLCN

SEQ ID NO: 44 - protein, 78 aa, Homo sapiens
VQCRMCHLQFPGEKCSRGRGICTATTEEACMVGRMFKRDGNPWLTFMGCLKNCADVKGIRWSVYLVNFRCCRSHDLCN

SEQ ID NO: 45 - protein, 74 aa, Homo sapiens
LKCNTCIYTEGWKCMAGRGTCIAKENELCSTTAYFRGDKHMYSTHMCKYKCREEESSKRGLLRVTLCCDRNFCN

SEQ ID NO: 46 - protein, 74 aa, Homo sapiens
QCITCHLRTRTDRCRRGFGVCTAQKGEACMLLRIYQRNTLQISYMVCQKFCRDMTFDLRNRTYVHTCCNYNYCN

SEQ ID NO: 47 - protein, 80 aa, Homo sapiens
IMCYECKKYHLGLCYGVMTSCSLKHKQSCAVENFYILTRKGQSMYHYSKLSCMTSCEDINFLGFTKRVELICCDHSNYCN

SEQ ID NO: 48 - protein, 73 aa, Homo sapiens
LLCYSCKAQVSNEDCLQVENCTQLGEQCWTARIRAVGLLTVISKGCSLNCVDDSQDYYVGKKNITCCDTDLCN

SEQ ID NO: 49 - protein, 78 aa, Homo sapiens
LKCYTCKEPMTSASCRTITRCKPEDTACMTTLVTVEAEYPFNQSPVVTRSCSSSCVATDPDSIGAAHLIFCCFRDLCN

SEQ ID NO: 50 - protein, 73 aa, Homo sapiens
IWCHQCTGFGGCSHGSRCLRDSTHCVTTATRVLSNTEDLPLVTKMCHIGCPDIPSLGLGPYVSIACCQTSLCN

SEQ ID NO: 51 - protein, 76 aa, Homo sapiens
LNCYTCAYMNDQGKCLRGEGTCITQNSQQCMLKKIFEGGKLQFMVQGCENMCPSMNLFSHGTRMQIICCRNQSFCN

SEQ ID NO: 52 - protein, 75 aa, Homo sapiens
LRCYTCKSLPRDERCNLTQNCSHGQTCTTLIAHGNTESGLLTTHSTWCTDSCQPITKTVEGTQVTMTCCQSSLCN

SEQ ID NO: 53 - protein, 75 aa, Homo sapiens
CVFCELTDSMQCPGTYMHCGDDEDCFTGHGVAPGTGPVINKGCLRATSCGLEEPVSYRGVTYSLTTNCCTGRLCN

SEQ ID NO: 54 - protein, 82 aa, Homo sapiens
LICRACNLSLPFHGCLLDLGTCQAEPGQYCKEEVHIQGGIQWYSVKGCTKNTSECFKSTLVKRILQLHELVTTHCCNHSLCN

SEQ ID NO: 55 - protein, 78 aa, Homo sapiens
IRCYCDAAHCVATGYMCKSELSACFSRLLDPQNSNSPLTHGCLDSLASTTDICQAKQARNHSGTTIPTLECCHEDMCN

SEQ ID NO: 56 - protein, 82 aa, Pongo pygmaeus
LAVFCHSGHSLQCYSCPNPTADCKTAVNCSSGFDACLITKAGLRVYNQCWKFEHCTFNEVTTRLKESELTYHCCKKDLCNIN

SEQ ID NO: 57 - protein, 82 aa, African Green Monkey
LAVFCHSGHSLQCYNCPNPTTDCKTAINCSSGFDTCLIARAGLQVYNQCWKFANCNFNDISTLLKESELQYFCCKKDLCNFN

SEQ ID NO: 58 - protein, 82 aa, Macaca fascicularis
LAVFCHSGHSLQCYNCPNPTTDCKTAINCSSGFDTCLIARAGLQVYNQCWKFANCNYNDISTLLKESELRYFCCKKDLCNFN

SEQ ID NO: 59 - protein, 80 aa, Papio hamadryas
LAVFCHSGHSLQCYNCPNPTTNCKTAINCSSGFDTCLIARAGLQVYNQCWKFANCNFNDISTLLKESELQYFCCKEDLCN

SEQ ID NO: 60 - protein, 82 aa, Simia trivirgata
LAVFCHSGNSLQCYSCPYPTTQCTMTTNCTSNLDSCLIAKAGSRVYYRCWKFEDCTFSRVSNQLSENELKYYCCKKNLCNFN

SEQ ID NO: 61 - protein, 82 aa, Callithrix marmoset
LAVFCHSGHSLQCYSCPYSTARCTTTTNCTSNLDSCLIAKAGLRVYYRCWKFEDCTFRQLSNQLSENELKYHCCRENLCNFN

SEQ ID NO: 62 - protein, 85 aa, Saimiri sciureus
LAVFCHSGNSLQCYSCPLPTMESMECTASTNCTSNLDSCLIAKAGSGVYYRCWKFDDCSFKRISNQLSETQLKYHCCKKNLCNVK

SEQ ID NO: 63 - protein, 82 aa, Rabbit
LAVFYSSDSSLMCYHCLLPSPNCSTVTNCTPNHDACLTAVSGPRVYRQCWRYEDCNFEFISNRLEENSLKYNCCRKDLCNGP

SEQ ID NO: 64 - protein, 83 aa, Pig
LAVLCHLGHSLQCYNCINPAGSCTTAMNCSHNQDACIFVEAVPPKTYYQCWRFDECNFDFISRNLAEKKLKYNCCRKDLCNKS

SEQ ID NO: 65 - protein, 82 aa, Rattus rattus
LAVLCSTGVSLRCYNCLDPVSSCKTNSTCSPNLDACLVAVSGKQVYQQCWRFSDCNAKFILSRLEIANVQYRCCQADLCNKS

SEQ ID NO: 66 - protein, 82 aa, Mus musculus
LAVFCSTAVSLKCYNCFQFVSSCKINTTCSPNLDSCLYAVAGRQVYQQCWKLSDCNSNYIMSRLDVAGIQSKCCQWGLCNKN

SEQ ID NO: 67 - protein, 83 aa, Mus musculus
LAVFCSTAVSLTCYHCFQPVVSSCNMNSTCSPDQDSCLYAVAGMQVYQRCWKQSDCHGEIIMDQLEETKLKFRCCQFNLCNKS

SEQ ID NO: 68 - protein, 77 aa, Pan troglodytes
LRCMQCKTNGDCRVEECALGQDLCRTTIVRMWEEGEELELVEKSCTHSEKTNRTLSYRTGLKITSLTEVVCGLDLCN

SEQ ID NO: 69 - protein, 77 aa, Macaca fascicularis
LRCMQCKSNGDCRVEECALGQDLCRTTIVRMWEEGEELELVEKSCTHSEKTNRTMSYRTGLKITSLTEVVCGLDLCN

SEQ ID NO: 70 - protein, 77 aa, African Green Monkey; identical to SEQ ID NO: 69

SEQ ID NO: 71 - protein, 77 aa, Simia trivirgata
LRCMQCNGHGNCRVEECALGQNLCRTTSVRHWEEGEEVEMVEKSCTHSEKTNRTMSFRTGVRITTLTEAVCGLDLCN

SEQ ID NO: 72 - protein, 77 aa, Rattus rattus
LRCIQCESNQDCLVEECALGQDLCRTTVLREWEDAEELEVVTRGCAHKEKTNRTMSYRMGSVIVSLTETVCATNLCN

SEQ ID NO: 73 - protein, 77 aa, Mus musculus
LQCMQCESNQSCLVEECALGQDLCRTTVLREWQDDRELEVVTRGCAHSEKTNRTMSYRMGSMIISLTETVCATNLCN

SEQ ID NO: 74 - protein, 77 aa, Bovine
LRCLQCENTTSCSVEECTPGQDLCRTTVLSVWEGGNEMNVVRKGCTHPDKTNRSMSYRAADQIITLSETVCRSDLCN

SEQ ID NO: 75 - protein, 85 aa, African Green Monkey
LECISCGSSDMSCERGRHQSLQCRSPEEQCLDMVTHWIQEGEEGRPKDDRHLRGCGYLPGCPGSNGFHNNDTFHFLKCCNTTKCN

SEQ ID NO: 76 - protein, 85 aa, Macaca fascicularis
LECISCGSSDMSCERGRHQSLQCRSPEEQCLDVVTHWIQEGEEGRPKDDRHLRGCGYLPSCPGSSGFHNNDTFHFLKCCNTTKCN

SEQ ID NO: 77 - protein, 85 aa, Pan troglodytes
LECISCGSSNMSCERGRHQSLQCRNPEEQCLDVVTHWIQEGEEGRPKDDRHLRGCGYLPGCPGSNGFHNNDTFHFLKCCNTTKCN

SEQ ID NO: 78 - protein, 85 aa, Simia trivirgata
LECISCGSSDMSCERGRHQSLQCTSPKEQCLDMVTHRTSEAEEGRPKDDHHIRGCGHLPGCPGIAGFHSEDTFHFLKCCNTTKCN

SEQ ID NO: 79 - protein, 82 aa, Bovine
LECASCSSTDLSCERGWDQTMQCLKSRDQCVDVITHRSLKENPGDERHIRGCGILPGCPGPTGFHNNHTFHFLRCCNTTKCN

SEQ ID NO: 80 - protein, 82 aa, Mus musculus
LECASCTSLDQSCERGREQSLQCRYPTEHCIEVVTLQSTERSLKDEDYTRGCGSLPGCPGTAGFHSNQTFHFLKCCNYTHCN

SEQ ID NO: 81 - protein, 82 aa, Rattus rattus
LECASCTSLDQSCERGREQSLQCRYPTEHCIEVVTLQSTERSVKDEPYTKGCGSLPGCPGTAGFHSNQTFHFLKCCNFTQCN

SEQ ID NO: 82 - protein, 81 aa, Pan troglodytes; identical to SEQ ID NO: 4

SEQ ID NO: 83 - protein, 80 aa, Macaca fascicularis
QCYSCKGNSTHGCSSEETFLIDCRGPMNQCLVATGTYEPKNQSYMVRGCVTASMCQRAHLGDAFSMHHINVSCCTESGCN

SEQ ID NO: 84 - protein, 80 aa, African Green Monkey
QCYSCKGNSTHGCSSEETFLIDCRGPMNQCLVATGTYEPKNQSYMVRGCVTASMCQRAHLGDAFSMNHINVFCCTESGCN

SEQ ID NO: 85 - protein, 80 aa, Simia trivirgata
RCYSCQGNSTHGCSSENTVLTDCRGPMNQCLEATGIYEPLSESYMVRGCATSSMCQHDHVSDAFSMSHIDVACCTENDCN

SEQ ID NO: 86 - protein, 80 aa, Bovine
QCYSCEGNGAHRCSSEETFLIDCRGPMNQCLEATGTKGLRNPSYTIRGCAAPSWCQSLHVAEAFDLTHVNVSCCTGSGCN

SEQ ID NO: 87 - protein, 81 aa, Rattus rattus
QCYSCEGNSTFGCSYEETSLIDCRGPMNQCLEATGLDVLGNRSYTVRGCATASWCQGSHVADSFQTHVNLSISCCNGSGCN

SEQ ID NO: 88 - protein, 81 aa, Mus musculus
QCYSCEGNNTLGCSSEEASLINCRGPMNQCLVATGLDVLGNRSYTVRGCATASWCQGSHVADSFPTHLNVSVSCCHGSGCN

SEQ ID NO: 89 - protein, 7 aa, Artificial
  Peptide from the RGD loop of eristostatin
VARGDWN

SEQ ID NO: 90 - protein, 9 aa, Artificial
  Peptide from the RGD loop of eristostatin
RVARGDWND

SEQ ID NO: 91 - protein, 11 aa, Artificial
  Peptide from the RGD loop of eristostatin
RVARGDWNDDY

SEQ ID NO: 92 - DNA, 73 bp, Artificial
  Oligo
cggccatggctcttcaatgctacaactgtcctaacccgactgtggcacgtggtgattggaattgtaaaaccgc

SEQ ID NO: 93 - DNA, 74 bp, Artificial
  Oligo
ggttttacaattccaatcaccacgtgccacagtcgggttaggacagttgtagcattgaagagccatggccggct

SEQ ID NO: 94 - DNA, 79 bp, Artificial
  Oligo
cggccatggctcttcaatgctacaactgtcctaacccgactcgcgtggcacgtggtgattggaatgactgtaaaaccgc

SEQ ID NO: 95 - DNA, 80 bp, Artificial
  Oligo
ggttttacagtcattccaatcaccacgtgccacgcgagtcgggttaggacagttgtagcattgaagagccatggccggct

SEQ ID NO: 96 - DNA, 79 bp, Artificial
  Oligo
cggcccttcaatgctacaactgtcctaacccgactcgcgtggcacgtggtgattggaatgacgactactgtaaaaccgc

SEQ ID NO: 97 - DNA, 80 bp, Artificial
  Oligo
ggttttacagtagtcgtcattccaatcaccacgtgccacgcgagtcgggttaggacagttgtagcattgaagggccggct

SEQ ID NO: 98 - DNA, 40 bp, Artificial
  Oligo
cgcgtctccgtgaagtggcacgtggtgattggaatcttac

SEQ ID NO: 99 - DNA, 36 bp, Artificial
  Oligo
gtaagattccaatcaccacgtgccacttcacggaga

SEQ ID NO: 100 - DNA, 46 bp, Artificial
  Oligo
cgcgtctccgtgaacgcgtggcacgtggtgattggaatgaccttac

SEQ ID NO: 101 - DNA, 42 bp, Artificial
  Oligo
gtaaggtcattccaatcaccacgtgccacgcgttcacggaga

SEQ ID NO: 102 - DNA, 52 bp, Artificial
  Oligo
cgcgtctccgtgaacgcgtggcacgtggtgattggaatgacgactaccttac

SEQ ID NO: 103 - DNA, 48 bp, Artificial
  Oligo
gtaaggtagtcgtcattccaatcaccacgtgccacgcgttcacggaga

SEQ ID NO: 104 - DNA, 56 bp, Artificial
  Oligo
ccggtactcacgaagtggcacgtggtgattggaataatcaatcttatatggtccgc

SEQ ID NO: 105 - DNA, 50 bp, Artificial
  Oligo
ggaccatataagattgattattccaatcaccacgtgccacttcgtgagta

SEQ ID NO: 106 - DNA, 62 bp, Artificial
  Oligo
ccggtactcacgaacgcgtggcacgtggtgattggaatgacaatcaatcttatatggtccgc

SEQ ID NO: 107 - DNA, 56 bp, Artificial
  Oligo
ggaccatataagattgattgtcattccaatcaccacgtgccacgcgttcgtgagta

SEQ ID NO: 108 - DNA, 68 bp, Artificial
  Oligo
ccggtactcacgaacgcgtggcacgtggtgattggaatgacgactacaatcaatcttatatggtccgc

SEQ ID NO: 109 - DNA, 62 bp, Artificial
  Oligo
ggaccatataagattgattgtagtcgtcattccaatcaccacgtgccacgcgttcgtgagta

SEQ ID NO: 110 - DNA, 51 bp, Artificial
  Oligo
tcgatgcatgcttaattactaaagccnnnnnnncaggtgtacaataaatgt

SEQ ID NO: 111 - DNA, 20 bp, Artificial
  Oligo
gttccagcggatccggatac

SEQ ID NO: 112 - protein, 87 aa, Artificial
  7-mer Library Variant
LAVFCHSGHSLQCYNCPNPTADCKTAVNCSSDFDACLITKAPSKRDHPQVYNKCWKFEHCNFNDVTTRLRENELTYYCCKKDLCNFN

SEQ ID NO: 113 - protein, 87 aa, Artificial
  7-mer Library Variant
LAVFCHSGHSLQCYNCPNPTADCKTAVNCSSDFDACLITKAPPRGDYPQVYNKCWKFEHCNFNDVTTRLRENELTYYCCKKDLCNFN

SEQ ID NO: 114 - protein, 87 aa, Artificial
  7-mer Library Variant
LAVFCHSGHSLQCYNCPNPTADCKTAVNCSSDFDACLITKAHRGDHPDQVYNKCWKFEHCNFNDVTTRLRENELTYYCCKKDLCNFN